SEQUENCE LISTING (37 sequences)

SEQ ID NO: 1 - DNA, 774 bp, Gallus gallus
  misc_feature (1)..(774): CDS
atggcgactccccccaagcgcgggcggctggagggcagggtgaagaagatcgccgtcgagggcaacatcgctgctgggaaatcaacgtttgtgaatattctcaaacaagctgatgagggatgggaagttgttcctgagcctgtagctagatggtgcaacgtccaacaaaactctgaagaggattgtgaggaactgactacatcacagaagagtggtggaaatgtgcttcagatgatgtacgagaaaccggagaggtggtctttcaccttccagatgtacgcgtgcctcagcaggattcgggctcagctcaaatccattgatggcaaactcagagaggcagagaatcctgtggtgttctttgagcgatctgtctatagtgacagatatatctttgcagctaatttatatgagtctgactgtatgaatgaaactgagtggactatttaccaagactggcacgactggatgaataagcagtttggtcaaagccttgcactggatggaattatttatctcagagccactcctgagaaatgcttaaataggatttacttgcgtggaagagatgaagagcaagaaattcctattgagtatctggagaagcttcactacaaacatgaaagctggcttcagcataagacactgcgaacagattttgaatatctacaagaaatacctattttaacattagatgttaatgaagacttcaaaggcaaaaaggacagatatgatcacatgactgaaaaggtgaaggaatttttgagcacattataa

SEQ ID NO: 2 - DNA, 798 bp, Gallus gallus
  misc_feature (1)..(798): CDS
atgtccgctcccgccaagaggcgctgccgcggccccgccgccgacctggactccagcttccagaagcgcctcaggaagatctccatcgagggcaatatcgcagctggaaagtctacgctcgtcaggctgctcgagaagcacagcgatgagtgggaggtgatccctgagcccatcgccaagtggtgcaacatccagaccagtgaggatgagtgcaaggaactttcaacatctcagaagagtggaggaaacctacttcaaatgctgtatgataaacctacaagatgggcttatacttttcagacctacgcctgcttgagccgagtaagggcacagctaaaacccatctcagccaagctgcatgaagcagaacatccagtgcaattttttgagagatctgtgtacagtgacaggtatgtgtttgcttctaacctgtttgaatctggaaacatcaatgaaacagagtgggctatctatcaggactggcactcatggcttttaaatcagtttcaatctgaaatagaactggatggcatcatctacctaaggaccacacctcagaaatgcatggaaagactacagaagaggggaagaaaagaggaggaaggaattgacctcgagtatttggaaaacctccactataaacatgagacctggctttatgaaaaaactatgagagttgattttgagaaccttaaagagattcccattctagttttggatgttaatgaagattttaaaaatgataaaattaaacaggagtacctgattgatgagatcaaatctttcctgacttcttaa

SEQ ID NO: 3 - DNA, 40 bp, Artificial Sequence
  Description of Artificial Sequence  Synthetic oligonucleotide primer
ttaggatccatggcgactccccccaagcgcgggcggctgg

SEQ ID NO: 4 - DNA, 37 bp, Artificial Sequence
  Description of Artificial Sequence  Synthetic oligonucleotide primer
ccggaattcttataatgtgctcaaaaattccttcacc

SEQ ID NO: 5 - DNA, 37 bp, Artificial Sequence
  Description of Artificial Sequence  Synthetic oligonucleotide primer
ttaggatccatgtccgctcccgccaagaggcgctgcc

SEQ ID NO: 6 - DNA, 39 bp, Artificial Sequence
  Description of Artificial Sequence  Synthetic oligonucleotide primer
ccggaattcttaagaagtcaggaaagatttgatctcatc

SEQ ID NO: 7 - DNA, 38 bp, Artificial Sequence
  Description of Artificial Sequence  Synthetic oligonucleotide primer
cgccgcggatccatggccaccccgcccaagagaagctg

SEQ ID NO: 8 - DNA, 33 bp, Artificial Sequence
  Description of Artificial Sequence  Synthetic oligonucleotide primer
ccggaattcttacaaagtactcaaaaactcttt

SEQ ID NO: 9 - DNA, 31 bp, Artificial Sequence
  Description of Artificial Sequence  Synthetic oligonucleotide primer
cgcggatccatggcttcgtaccccggccatc

SEQ ID NO: 10 - DNA, 33 bp, Artificial Sequence
  Description of Artificial Sequence  Synthetic oligonucleotide primer
ccggaattcttagttagcctcccccatctcccg

SEQ ID NO: 11 - DNA, 38 bp, Artificial Sequence
  Description of Artificial Sequence  Synthetic oligonucleotide primer
tccctcgaggccaccatggcgactccccccaagcgcgg

SEQ ID NO: 12 - DNA, 35 bp, Artificial Sequence
  Description of Artificial Sequence  Synthetic oligonucleotide primer
gaagatcttcataatgtgctcaaaaattccttcac

SEQ ID NO: 13 - DNA, 37 bp, Artificial Sequence
  Description of Artificial Sequence  Synthetic oligonucleotide primer
acgcgtcgacgccaccatgtccgctcccgccaagagg

SEQ ID NO: 14 - DNA, 36 bp, Artificial Sequence
  Description of Artificial Sequence  Synthetic oligonucleotide primer
cggggatcctcaagaagtcaggaaagatttgatctc

SEQ ID NO: 15 - DNA, 41 bp, Artificial Sequence
  Description of Artificial Sequence  Synthetic oligonucleotide primer
ccgctcgaggccaccatggccaccccgcccaagagaagctg

SEQ ID NO: 16 - DNA, 33 bp, Artificial Sequence
  Description of Artificial Sequence  Synthetic oligonucleotide primer
gaagatcttcacaaagtactcaaaaactctttg

SEQ ID NO: 17 - DNA, 41 bp, Artificial Sequence; identical to SEQ ID NO: 15
  Description of Artificial Sequence  Synthetic oligonucleotide primer

SEQ ID NO: 18 - DNA, 33 bp, Artificial Sequence; identical to SEQ ID NO: 16
  Description of Artificial Sequence  Synthetic oligonucleotide primer

SEQ ID NO: 19 - DNA, 40 bp, Artificial Sequence; identical to SEQ ID NO: 3
  Description of Artificial Sequence  Synthetic oligonucleotide primer

SEQ ID NO: 20 - DNA, 37 bp, Artificial Sequence; identical to SEQ ID NO: 4
  Description of Artificial Sequence  Synthetic oligonucleotide primer

SEQ ID NO: 21 - DNA, 46 bp, Artificial Sequence
  Description of Artificial Sequence  Synthetic oligonucleotide primer
caccttccagatgtacgtgtgcctcagcatgattcgggctcagctc

SEQ ID NO: 22 - DNA, 46 bp, Artificial Sequence
  Description of Artificial Sequence  Synthetic oligonucleotide primer
gagctgagcccgaatcatgctgaggcacacgtacatctggaaggtg

SEQ ID NO: 23 - DNA, 42 bp, Artificial Sequence
  Description of Artificial Sequence  Synthetic oligonucleotide primer
ctttgagcgatctgtctatagtgccagatatatctttgcagc

SEQ ID NO: 24 - DNA, 42 bp, Artificial Sequence
  Description of Artificial Sequence  Synthetic oligonucleotide primer
gctgcaaagatatatctggcactatagacagatcgctcaaag

SEQ ID NO: 25 - DNA, 33 bp, Artificial Sequence
  Description of Artificial Sequence  Synthetic oligonucleotide primer
ggtggtctttcacctaccagatgtacgcgtgcc

SEQ ID NO: 26 - DNA, 33 bp, Artificial Sequence
  Description of Artificial Sequence  Synthetic oligonucleotide primer
ggcacgcgtacatctggtaggtgaaagaccacc

SEQ ID NO: 27 - DNA, 34 bp, Artificial Sequence
  Description of Artificial Sequence  Synthetic oligonucleotide primer
cgcggatccatggccaagagcccactcgagggcg

SEQ ID NO: 28 - DNA, 37 bp, Artificial Sequence
  Description of Artificial Sequence  Synthetic oligonucleotide primer
ccggaattcttacagattctttacaaaggtgtttacc

SEQ ID NO: 29 - protein, 260 aa, Homo sapiens [sequence omitted]

SEQ ID NO: 30 - protein, 260 aa, Mus musculus
MATPPKRFCPSPSTSSEGTRIKKISIEGNIAAGKSTFVNILKQASEDWEVVPEPVARWCNVQSTQEEFEELTTSQKSGGNVLQMMYEKPERWSFTFQSYACLSRIRAQLASLNGKLKDAEKPVLFFERSVYSDRYIFASNLYESDCMNETEWTIYQDWHDWMNSQFGQSLELDGIIYLRATPEKCLNRIYLRGRNEEQGIPLEYLEKLHYKHESWLLHRTLKTSFDYLQEVPVLTLDVNEDFKDKHESLVEKVKEFLSTL

SEQ ID NO: 31 - protein, 260 aa, Rattus norvegicus
MATPPKRFCSSPSTSSEGTRIKKISIEGNIAAGKSTFVNILKQVCEDWEVVPEPVARWCNVQSTQDEFEELTTSQKSGGNVLQMMYEKPERWSFIFQSYACLSRIRAQLASLNGSLRDAEKPVLFFERSVYSDRYIFASNLYESDCMNETEWTIYQDWHDWMNSQFGQSLELDGIIYLRATPEKCLNRIYIRGRDEEQGIPLEYLEKLHYKHESWLLHRTLKTNFEYLQEVPILTLDVNLDFKDKEESLVEKVKEFLSTT

SEQ ID NO: 32 - protein, 264 aa, Xenopus laevis
MATPPKRMCHSPVFNNSFEKRVKKLSIEGNIAAGKSTFVRILEKASDEWEVVPEPIAKWCNVQTTENENEELSTSQKSGGNLLQMLYDKPTRWAYTFQTYACLSRVRAQLKTPSPKLLEAEHPVQFFERSVYSDRYIFASSLFEFQNINETEWAIYQDWHTWFLNQFESDIDLDGIIYLRATPEKCMDRLHTRGREEEQGIQLEYLESLHYKHESWLYDRTMSVDFENLQHMPVMVLDVNEDFKYDKIKQEALLDKVKEFLASL

SEQ ID NO: 33 - protein, 264 aa, Danio rerio
MATPPKRLCSSFDADLSFEKRAMKVSIEGNIAAGKSTFVRLLERASEEWEVIPEPIGKWCNVQTTENEYEELSTSQKSGGNLLQMLYDKPSRWSYTFQTYACLSRVRSQLQPPSAKLQQAEKPVQFFERSVYSDRYVFASNLFESGDLNETEWAIYQDWHSWLLTQFESQIELDAMIYLRADPERCMQRLQFRGREEEQGIPLDYLEKLHYKHECWLYNQTTKLDFEYLKDLPILILDVNEDFKNDRIKQEGVIDKVKEFLNSL

SEQ ID NO: 34 - DNA, 42 bp, Artificial Sequence
  Description of Artificial Sequence  Synthetic oligonucleotide derived from Gallus gallus
aaggaatttttgagcacattaggatccatggcgactcccccc

SEQ ID NO: 35 - protein, 14 aa, Artificial Sequence
  Description of Artificial Sequence  Synthetic peptide derived from Gallus gallus
KEFLSTLGSMATPP

SEQ ID NO: 36 - DNA, 24 bp, Gallus gallus
aaggaatttttgagcacattataa

SEQ ID NO: 37 - protein, 7 aa, Gallus gallus
KEFLSTL

(19) United States
(12) Patent Application Publication
(10) Pub. No.: US 2007/0248543 A1
(43) Pub. Date: Oct. 25, 2007

(54) Chicken Deoxycytidine and Deoxyadenosine Kinase Enzymes and Their Use

(75) Inventor: Zoran Gojkovic, Holte (DK)

Correspondence Address: BIRCH STEWART KOLASCH & BIRCH, PO BOX 747, FALLS CHURCH, VA, 22040-0747, US

(73) Assignee: ZGene A/S, Lyngby (DK)

(21) Appl. No.: 11/629,526

(22) PCT Filed: Jun. 22, 2005
(86) PCT No.: PCT/DK05/00421

§ 371 Date: Dec. 14, 2006

Related U.S. Application Data
(60) Provisional application No. 60/583,608, filed on Jun. 30, 2004.

(30) Foreign Application Priority Data
Jun. 30, 2004 (DK) PA 2004 01036
Apr. 12, 2005 (DK) PA 2005 00524

(51) Int. Cl. A61K 31/7052; A61K 39/395; A61K 48/00; A61K 49/00; C07H 21/04; C07K 14/00; C12N 1/21; C12N 15/00; C12N 5/06; C12P 1/04

(52) U.S. Cl. 424/9.1; 424/130.1; 424/93.47; 424/94.5; 435/193; 435/320.1; 435/366; 435/375; 435/6; 435/69.1; 514/44; 536/23.1; 536/25.3

(57) ABSTRACT

The application relates to the field of suicide gene therapy using expression vectors encoding a deoxynucleotide kinase capable of converting prodrugs into cytotoxic drugs. In particular, Chicken deoxycytidine kinase and eukaryotic deoxyadenosine kinase polypeptides and nucleotides encoding such polypeptides and a procedure for producing such polypeptides by recombinant techniques are disclosed. Also disclosed are methods for utilizing such polypeptides for the treatment of malignancies and viral infections, methods of sensitising cells to prodrugs, and methods of inhibiting pathogenic agents in warm-blooded animals using said dCKs and dAKs.

The present application claims the benefit of U.S. Ser. No. 60/583,608 filed 30 Jun. 2004, which is incorporated by reference in its entirety. It claims priority from Danish patent applications no. PA 2004 01036, filed 30 Jun. 2004, and PA 2005 00524, filed 12 Apr. 2005. All references cited in those applications and in the present application are hereby incorporated by reference in their entirety.

### TECHNICAL FIELD

The application relates to the field of suicide gene therapy using expression vectors encoding a deoxynucleotide kinase capable of converting prodrugs into cytotoxic drugs. Chicken deoxycytidine kinase and deoxyadenosine kinase polypeptides and nucleotides encoding such polypeptides and a procedure for producing such polypeptides by recombinant techniques are disclosed. Also disclosed are methods for utilizing such polypeptides for the treatment of malignancies and viral infections, methods of sensitising cells to prodrugs, and methods of inhibiting pathogenic agents in warm-blooded animals using said dCKs and dAKs.

This invention relates to newly identified polynucleotides, polypeptides encoded by such polynucleotides, the use of such polynucleotides and polypeptides, as well as the production of such polynucleotides and polypeptides. More particularly, the polypeptides of the present invention are chicken (Gallus gallus) deoxycytidine kinase 1 and 2, referred to as “GgcCK1 and GgdCK2”. The invention also relates to medical use of such polypeptides.

### BACKGROUND ART

DNA is made of four deoxyribonucleoside triphosphates, provided by the de novo and the salvage pathway. The key enzyme of the de novo pathway is ribonucleotide reductase, which catalyses the reduction of the 2′-OH group of the nucleoside diphosphates, and the key salvage enzymes are the deoxyribonucleoside kinases, which phosphorylate deoxyribonucleosides to the corresponding deoxyribonucleoside monophosphates.

Deoxycytidine kinase is responsible for the phosphorylation of several deoxyribonucleosides and their analogs. The enzyme has been shown to have broad substrate specificity and plays a physiological role in the maintenance of the normal deoxyribonucleotide pools. Deoxycytidine kinase is-also a key enzyme in the phosphorylation of a variety of antineoplastic and antiviral nucleoside analogs including 1-D-arabinofuranosylcytosine and dideoxycytidine (Ullman, B. et al, J.Biol.Chem., 263:12391-12396 (1988)), and deficiency of deoxycytidine kinase actively mediates resistance to these drugs. The enzyme is allosterically regulated by several deoxyribonucleotides and preferentially uses ATP as a phosphate donor for the phosphorylization of deoxycytidine (Ikeda, S. et al., Bio.Chem., 27:8648-8652 (1988)).

The deoxycytidine kinase protein has a number of substrates including cytosine arabinoside, deoxyguanosine, deoxyadenosine, cytidine, 2-chloro-adenosine and dideoxycytidine. Deoxycytidine kinase is the rate limiting step in the activation of the chemotherapeutic agent cytosine arabinoside to its 5′ triphosphate (Mejer, J., Scand.J.Clin.Lab.lnvest., 42:401-406 (1982)). Other clinically important chemotherapeutic agents for which deoxycytidine kinase catalyzes the initial activation of is ara-C, 2-fluoro-9-D-arabinofuranosyladenine, and dideoxycytidine (Kufe, D. W. and Spriggs, D. R., Semin.Oncol., 12:34-48 (1985)).

Human deoxycytidine kinase has been partially purified from variety of human tissues such as lymphocytes, spleen, T-lymphoblasts, and myeloblasts. (Baxter, A. et al., Bio.Chem.J., 173:1005-1008 (1978)).

Deoxyadenosine kinase (dAK, EC 2.7.1.76) was for a long time believed to be the crucial enzyme responsible for the phosphorylation of dAdo but has not been found in any eukaryots. The controversy about the identity of the deoxynucleoside kinases that are responsible for dAdo phosphorylation resulted in numerous yet inconclusive data. Mammals contain four different deoxyribonucleoside kinases: the cytoplasmic thymidine kinase 1 (TK1, EC 2.7.1.21) and deoxycytidine kinase (dCK, EC 2.7.1.74), and the mitochondrial enzymes thymidine kinase 2 (TK2, EC 2.7.1.21) and deoxyguanosine kinase (dGK, EC 2.7.1.113). All these enzymes have distinct but overlapping specificities. TK1 phosphorylates only thymidine (Thd) and deoxyuridine (dUrd), TK2 phosphorylates Thd, dUrd and deoxycytidine (dCyd), while substrates for dGK are deoxyadenosine (aAdo) and deoxyguanosine (dGuo). dCK is the only enzyme which can phosphorylate both pyrimidine (dCyd) and purine (dAdo and dGuo) deoxyribonucleosides. Therefore in mammalian cells dAdo can be phosphorylated either by dCK in cytoplasm or by dGK in mitochondria. The general agreement today is that mammalian cells do not have a designated dAK enzyme for dAdo phosphorylation. This is supported by human genome sequencing data where no dAK gene is present.

Several EST sequences from chicken have been annotated as putative deoxycytidine kinase (dCK). However, up to this date no full ORF has been determined and no experimental work towards characterisation, properties, localisation, use or biological function of chicken kinases has yet been accomplished.

### SUMMARY OF THE INVENTION

It is an object of the present invention to provide chicken deoxycytidine and deoxyadenosine kinases useful for converting nucleoside analogs into toxic substances, and useful for converting nucleosides into monophosphates. In particular it is an object of the invention to provide such chicken dCK and dAK for medical use.

It is a further object to provide pharmaceutical compositions making use of the properties of said chicken-derived deoxycytidine kinases.

Our study showed that extracts from chicken cells efficiently phosphorylated all the natural deoxyribonucleosides. This suggested presence of various deoxyribonucleside kinases as reported for mammalian cells. Indeed EST sequences have revealed the existence of TK1, TK2, and dGK kinases. However, in addition to a novel chicken dCK we also discovered an additional kinase, proved to be dAK, which was never before reported in any eukaryotic organism. Chickens therefore have five different deoxyriboncleoside kinases. This enzyme is closely related to dCK and both genes have a common progenitor. However, chicken dAK showed unique properties with regards for both substrate specificity and gene organization. Gallus gallus deoxyadenosine kinase (GgdAK) showed preference for dAdo and dAdo analogs, whereas Gallus gallus dCK1 prefers dcyd and dcyd analogs as substrates. Therefore, dAK activity observed in chicken cells is unique and must represent a long missing dAK enzyme. Both enzymes have very relaxed substrate specificity and are able to phosphorylate dCyd, dAdo and dGuo, as well as several of their analogs. Until now dAK enzymes were reported only in microorganisms such as bacteria and mycoplasma.

In the following, both chicken enzymes are referred to as deoxycytidine kinases and are designated Gallus gallus deoxycytidine kinase 1, GgdCK1, and Gallus gallus deoxycytidine kinase 2, GgdCK2. The designation “chicken deoxycytidine kinase” covers chicken kinases belonging to the group of dCK/dGK/TK2-like family of deoxyribonucleoside kinases and being capable of phosphorylating dAdo, dCyd and dGuo. Deoxyadenosine kinases (dAKs) share these properties with dCKs but have a higher Kcat/Km ratio for dAdo than for dcyd and dGuo. Preferably, a dAK also has a higher Kcat/Km ratio for adenosine analogues compared to cytidine analogues.

When reference is made to a Gallus gallus deoxyadenosine kinase enzyme this reference is to GgdCK2 and to sequence variants of GgdCK2 with dAK activity.

The present invention provides novel polypeptides which are GgdCK1 and GgdCK2, as well as biologically active and therapeutically useful fragments, analogs and derivatives thereof. The invention also provides mutants of GgdCK1 and GgdCK2 with improved kinetic properties compared to the wild-type enzyme.

In a first aspect the invention relates to an isolated eukaryotic deoxyadenosine kinase enzyme (EC 2.7.1.76). This aspect is based on the current inventors' identification of the first eukaryotic dAK ever. In a preferred aspect, the dAK enzyme is derived from a vertebrate. Even more preferably the dAK is derived from an avian species.

In a further aspect the invention relates to an isolated polynucleotide encoding a Gallus gallus deoxycytidine kinase or a functional analogue thereof.

In a preferred embodiment of the first aspect, the isolated polynucleotide is selected from the group consisting of:

(a) a polynucleotide encoding a polypeptide having the amino acid sequence of SEQ ID No. 2,

(b) a polynucleotide having the nucleotide sequence of SEQ ID No 1,

(c) a polynucleotide encoding a dCK polypeptide, said dCK polypeptide having at least 80% sequence identity to SEQ ID No2,

(d) a polynucleotide encoding a dCK polypeptide, said polynucleotide having at least 80% sequence identity to the coding sequence of SEQ ID No 1,

(e) a polynucleotide capable of hybridising to a complement of SEQ ID No. 1, said polynucleotide encoding a dCK, and

(f) the complement of a through e.

In another preferred embodiment of the first aspect, the isolated polynucleotide is selected from the group consisting of:

(a) a polynucleotide encoding a polypeptide having the amino acid sequence of SEQ ID No. 4,

(b) a polynucleotide having the nucleotide sequence of SEQ ID No. 3

(c) a polynucleotide encoding a dCK polypeptide, said dCK polypeptide having at least 60% sequence identity to SEQ ID No. 4.

(d) a polynucleotide encoding a dCK polypeptide, said polynucleotide having at least 60% sequence identity to SEQ ID No. 3,

(e) a polynucleotide capable of hybridising to a complement of SEQ ID No. 3, said polynucleotide encoding a dCK, and

(f) the complement of a through e.

In a further aspect the invention relates to a vector comprising the polynucleotide of the invention. The polynucleotide may be a DNA.

In a further aspect the invention relates to an isolated host cell genetically engineered with the vector of the invention.

Furthermore the invention relates to a packaging cell line capable of producing an infective virion comprising the virus vector of the invention.

The invention also provides a process for producing a dCK polypeptide comprising culturing a host cell of the invention in vitro and recovering the expressed dCK from the culture.

In a further aspect the invention relates to an isolated deoxycytidine kinase derived from Gallus gallus or a functional analogue thereof.

In a preferred embodiment of this aspect, the isolated deoxycytidine kinase polypeptide comprises a polypeptide selected from the group consisting of:

(a) a polypeptide having the amino acid sequence of SEQ ID No 2, and

(b) a dCK polypeptide having at least 80 % sequence identity to SEQ ID No2

In another preferred embodiment of this aspect, the isolated deoxycytidine kinase polypeptide comprises a polypeptide selected from the group consisting of:

(a) a polypeptide having the amino acid sequence of SEQ ID No. 4, and

(b) a dCK polypeptide having at least 60% sequence identity to SEQ ID No. 4.

In a further aspect, the invention relates to a pharmaceutical composition comprising the polypeptide of the invention, the expression vector of the invention, the host cell of the invention, or the packaging cell line of the invention, and a pharmaceutically acceptable carrier or diluent.

In a further aspect, the invention relates to the use of the polypeptide of the invention for the preparation of a medicament, to use of the expression vector of the invention for the preparation of a medicament, to use of the host cell of the invention for the preparation of a medicament, and to use of the packaging cell line of the invention, for the preparation of a medicament. Preferably, the medicament is for the treatment of cancer. In another preferred embodiment the medicament is for the treatment of graft versus host disease (GVHD). In another embodiment, the use is for the treatment of a viral, bacterial, or parasite infection.

The invention also provides pharmaceutical articles comprising a source of a Gallus gallus derived dCK or a functional analog thereof and a nucleoside analogue for the simultaneous, separate or successive administration in cancer therapy.

In another aspect, the invention relates to a method of sensitising a cell to a nucleoside analogue prodrug, which method comprises the steps of:

(i) transfecting or transducing said cell with a polynucleotide sequence of the invention encoding a deoxycytidine kinase enzyme capable of promoting the conversion of said prodrug into a cytotoxic drug, or with an expression vector comprising said polynucleotide sequence; and

(ii) delivering said nucleoside analogue prodrug to said cell; wherein said cell is more sensitive to said cytotoxic drug than to said nucleoside analogue prodrug.

In a still further aspect, there is provided a method of inhibiting a pathogenic agent in a warm-blooded animal, which method comprises administering to said animal a polynucleotide of the invention or expression vector of the invention. By administering the GgdCK polynucleotides of the invention to an animal cell, the nucleotide pool in that cell is changed and the cells are rendered more sensitive to treatment. The change in nucleotide pool may in itself cause the cell to go into apoptosis. Cells already infected with virus have changed nucleotide pools and expressing a GgdCK of the present invention in such cells may lead to apoptosis of the virus-infected cell. Normal cells are much less sensitive to this kind of treatment.

In a further aspect, the invention provides an antibody against the polypeptide of the invention.

Furthermore, the invention relates to a method of phosphorylating a nucleoside or nucleoside analogue comprising the steps of.

(i) subjecting the nucleoside or nucleoside analogue to the action of a Gallus gallus dCK, and

(j) recovering the phorphorylated nucleoside or nucleoside analogue.

The present invention also relates to a method for the treatment of a patient having need of dCK comprising: administering to the patient a therapeutically effective amount of the polypeptide of the invention, wherein the polypeptide is administered by providing to the DNA encoding said polypeptide and expressing said polypeptide in vivo.

In a further aspect, the invention relates to a process for identifying compounds effective as antagonists to Gallus gallus dCKs comprising combining the polypeptide of the invention, a compound to be screened and a reaction mixture containing deoxyribonucleosides; and determining the ability of the compound to inhibit the phosphorylation of the deoxyribonucleosides.

In accordance with yet a further aspect of the present invention, there is provided a process for utilizing such polypeptide, or polynucleotide encoding such polypeptide for therapeutic purposes, for example, to phosphorylate deoxyribonucleosides to activate specific anti-cancer and anti-viral drugs. Due to the broad substrate specificity, these kinases can be used for a wide range of applications, including medical use.

In a still further aspect the invention provides methods of phosphorylating nucleosides or nucleoside analogs, comprising the steps of subjecting the nucleosides or nucleoside analogs to the action of the Chicken deoxyribonucleoside kinase enzymes of the invention, and recovering the phosphorylated nucleosides or nucleoside analogs.

The uses stem from the broad substrate specificity and/or the improved kinetic properties of the enzymes provided with the present invention.

In a further aspect the invention provides a method of non-invasive nuclear imaging of transgene expression of a chicken deoxycytidine kinase enzymes of the invention in a cell or subject.

For the development of effective clinical suicide gene therapy protocols, a non-invasive method to assay the extent, the kinetics and the spatial distribution of transgene expression is essential. Such imaging methods allow investigators and physicians to assess the efficiency of experimental and therapeutic gene transfection protocols and would enable early prognosis of therapy outcome.

Radionuclide imaging techniques like single photon emission computed tomography (SPECT) and positron emission tomography (PET), which can non-invasively visualize and quantify metabolic processes in vivo, are being evaluated for repetitive monitoring of transgene expression in living animals and humans. Transgene expression can be monitored directly by imaging the expression of the therapeutic gene itself, or indirectly using a reporter gene that is coupled to the therapeutic gene. Various radiopharmaceuticals have been developed and are now being evaluated for imaging of transgene expression.

These and other aspects of the present invention should be apparent to those skilled in the art from the teachings herein.

### BRIEF DESCRIPTION OF THE DRAWINGS

FIG. 1 illustrates the cDNA sequence and the corresponding deduced amino acid sequence of GgdCK-1 and GgdCK-2 polypeptides. The standard 1 letter abbreviations for amino acids is used.

FIG. 1a shows the cDNA sequence for Gallus gallus deoxycytidine kinase 1 (GgdCK1) having SEQ ID No.1, and the deduced amino acid sequence (SEQ ID No. 2).

FIG. 1b shows the cDNA sequence for Gallus gallus deoxycytidine kinase 2 (GgdCK2 or GgdAK) having SEQ ID No. 3, and the deduced amino acid sequence (SEQ ID No. 4).

FIG. 2 illustrates the amino acid sequence homology between chicken deoxycytidine kinases (SEQ ID No 2 and 4) and mammalian dCKs, wherein the black areas represent amino acid residues which are the same between the different sequences while shaded areas represent amino acid residues which are similar between the different sequences. The following mammalian sequences were used: Human dCK (ACCN. P27707, SEQ ID No. 31), mouse dCK (ACCN. P43346, SEQ ID No. 32) and rat dCK (ACCN. NP_{—}077072, SEQ ID No. 33). Residues interacting with substrate, as determined within the crystal structure of human dCK, are marked with arrows. P-loop, nuclear localisation signal, insert region, ERS motif and LID region are shown. ClustalX 1.81 (Jeanmougin, F.; Thompson, J. D.; Gouy, M.; Higgins, D. G.; Gibson, T. J. Multiple sequence alignment with Clustal X Trends Biochem.Sci. 23: 403-405.) was used for multiple sequence alignment.

FIG. 3 illustrates the connecting part linking the two gene sequences in the double gene (vector pZG634, example 7). The connecting part is represented with its coding amino acid sequence compared to chicken dCK1 wild type nucleotide and amino acid sequence. The BamHI restriction site is underlined and the start codon atg beside it is illustrated in bold letters.

FIG. 4 shows alignment of chicken doxycytidine kinases (SEQ ID No. 2 and 4) using a default settings of ClustalX 1.81 (Jeanmougin, F.; Thompson, J. D.; Gouy, M.; Higgins, D. G.; Gibson, T. J. Multiple sequence alignment with Clustal X Trends Biochem.Sci. 23: 403-405.) wherein the black areas represent amino acid residues which are the same between the different sequences while shaded areas represent amino acid residues which are similar between the different sequences.

FIG. 5 Distance tree derived from corrected-distance matrix. Multisequence alignment was made with the ClustalW 1.81 program. The program used were Kimura Distance and TreeCon. The sequences were: Chicken dCK (SEQ ID No. 2), Chicken dAK (SEQ ID No. 4), Human dCK (Genbank P27707, SEQ ID No. 31), rat dCK (Genbank NP_{—}077072, SEQ ID No 33), Xenopus dCK-D (Genbank AA064436), Zebrafish dCK (Genbank: AAH83277), Xenopus dAK (Genbank: M064435, SEQ ID No. 34), and Zebrafish dAK (Genbank: M064438, SEQ ID No. 35).

FIG. 6. illustrates the amino acid sequence homology between eukaryotic deoxyadenosine kinases and human dCK, wherein the black areas represent amino acid residues which are the same between the different sequences while shaded areas represent amino acid residues which are similar between the different sequences. The sequences are: Chicken dAK (SEQ ID No. 4), Xenopus dAK (Genbank: M064435, SEQ ID No. 34), Zebrafish dAK (Genbank: M064438, SEQ ID No. 35), and Human dCK (Genbank P27707, SEQ ID No. 31). Regions of high homology present in dAKs but not in dCK are boxed.

### DETAILED DISCLOSURE OF THE INVENTION

### DEFINITIONS

Deoxyribonucleoside Kinase.

DNA is made of four deoxyribonucleoside triphosphates, provided by the de novo and the salvage pathway. The key enzyme of the de novo pathway is ribonucleotide reductase, which catalyses the reduction of the 2′-OH group of the nucleoside diphosphates, and the key salvage enzymes are the deoxyribonucleoside kinases, which phosphorylate deoxyribonucleosides to the corresponding deoxyribonucleoside monophosphates. According to the present invention a deoxyribonucleoside kinase is an enzyme capable of phosophorylating at least one deoxyribonucleoside or deoxyribonucleoside analogue. A multisubstrate deoxyribonucleoside kinase is capable of phosphorylating all four deoxyribonucleosides to the corresponding monophosphates.

Deoxycytidine kinases (dCK) are structurally related to human dCK and are capable of phosphorylating dCyd, dGuo and dAdo. Deoxyadenosine kinases share these properties with dCKs but have a higher Kcat/Km ratio for dAdo than for dGuo and dCyd. Preferably, dAKs also have higher Kcat/Km ratios for adenosine analogues than for cytidine analogues.

Nucleoside Analogue.

A nucleoside analogue is defined as a compound comprising a deoxyribonucleoside structure, which compound is substituted in relation to a naturally occurring deoxyribonucleoside either on the deoxyribose part, or in the purine or pyrimidine ring. A nucleoside analogue is essentially non-toxic in its non-phosphorylated (nucleoside) state. Analogs of the naturally occurring nucleosides are usually administered as prodrugs, e.g. unphosphorylated, as the omission of the negative charges from the phosphate groups allows effective transport of the analog into the cell. Once prodrugs are converted into a potent cytotoxic metabolite they inhibit or disrupt DNA synthesis. The treated cells subsequently die via necrotic or apoptotic pathways.

Sequence Identity:

In the context of this invention “identity” is a measure of the degree of homology of amino acid sequences. In order to characterize the identity, subject sequences are aligned so that the highest order homology (match) is obtained. Based on these general principles the “percent identity” of two amino acid sequences may be determined using the BLASTP algorithm [Tatiana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site, and using the default settings suggested here (i.e. Matrix=Blosum62; Open gap=11; Extension gap=1; Penalties gap x_dropoff=50; Expect=10; Word size=3; Filter on). Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment and is therefore preferred for comparison to full length proteins. AlignO does not penalise to gaps in the end of the sequences. When utilizing the ALIGN or AlignO program for comparing amino acid sequences, a BLOSUM50 substitution matrix with gap opening/extension penalties of −12−/2 is preferably used.

In the context of this invention, “identity” is a measure of the degree of homology of nucleotide sequences. In order to characterize the identity, subject sequences are aligned so that the highest order homology (match) is obtained. Based on these general principles, the “percent identity” of two nucleic acids may be determined using the BLASTN algorithm [Tatiana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site, and using the default settings suggested here (i.e. Reward for a match=1; Penalty for a mismatch=−2; Strand option=both strands; Open gap=5; Extension gap=2; Penalties gap x_dropoff=50; Expect=10; Word size=11; Filter on).

Hybridisation Conditions:

Suitable experimental conditions for determining hybridisation at low, medium, or high stringency conditions, respectively, between a nucleotide probe and a homologous DNA or RNA sequence, involves pre-soaking of the filter containing the DNA fragments or RNA to hybridise in 5×SSC [Sodium chloride/Sodium citrate; cf. Sambrook et al.; Molecular Cloning: A Laboratory Manual, 2^{nd} Ed., Cold Spring Harbor Lab., Cold Spring Harbor, N.Y. 1989] for 10 minutes, and prehybridization of the filter in a solution of 5×SSC, 5×Denhardt's solution [cf.

Sambrook et al; Op cit.], 0.5% SDS and 100 pg/ml of denatured sonicated salmon sperm DNA [cf. Sambrook et al.,; Op cit.], followed by hybridisation in the same solution containing a concentration of 10 ng/ml of a random-primed [Feinberg A P & Vogelstein B; Anal. Biochem. 1983 132 6-13], ³²P-dCTP-labeled (specific activity>1×10⁹ cpm/pg) probe for 12 hours at approximately 45° C.

The filter is then washed twice for 30 minutes in 2×SSC, 0.5% SDS at a temperature of at least 55° C. (low stringency conditions), more preferred of at least 60° C. (medium stringency conditions), still more preferred of at least 65° C. (medium/high stringency conditions), even more preferred of at least 70° C. (high stringency conditions), and yet more preferred of at least 75° C. (very high stringency conditions).

Molecules to which the oligonucleotide probe hybridises under these conditions may be labelled to detect hybridisation. The complementary nucleic acids or signal nucleic acids may be labelled by conventional methods known in the art to detect the presence of hybridised oligonucleotides. The most common method of detection is the use of autoradiography with e.g. ³H, ¹²⁵I, ³⁵S, ¹⁴C, or ³²P-labelled probes, which may then be detected using an x-ray film. Other labels include ligands, which bind to labelled antibodies, fluorophores, chemoluminescent agents, enzymes, or antibodies, which can then serve as specific binding pair members for a labelled ligand.

Eukaryotic Deoxyadenosine Kinase

By the present invention there is provided an isolated eukaryotic deoxyadenosine kinase enzyme (EC 2.7.1.76).

As the present inventors have proven the existence, structure and properties of a dAK in Gallus gallus, it also becomes possible to isolate dAKs from other vertebrates and to distinghuish these from the dCK from the same species. With the presence of a dAK in the avian species Gallus gallus, it is evident that other avian species also have dAK enzymes. Birds are related to reptiles, which are also expected to contain a dedicated dAK gene. In addition, dAKs have been found in zebrafish and in Xenopus laevis. Based on these findings it is expected that dAKs can be found in Amphibiae and in fish. dAKs from these species are also included within the scope of the present invention.

Deoxyadnosine kinase enzymes from other eukaryots may be identified by searching expressed sequence tag libraries with translated Blast search tool using SEQ ID No. 4 as query

Preferably, the dAK enzyme of the invention is selected from the group consisting of:

(a) a dAK enzyme having the amino acid sequence of SEQ ID No. 4,

(b) a dAK enzyme having an amino acid sequence that is at least 60% sequence identity to SEQ ID No. 4,

(c) a dAK enzyme encoded by a polynucleotide having at least 60% sequence identity to SEQ ID No. 3, and

(d) a dAK enzyme encoded by a polynucleotide capable of hybridising to a polynucleotide having the complement of SEQ ID No. 3

More preferably the dAK enzyme of the invention has an amino acid sequence that has at least 65% sequence identity to SEQ ID No 4, preferably at least 70%, more preferably at least 75%, more preferably at least 80%, more preferably at least 85%, more preferably 90%, more preferably at least 95%, more preferably at least 98%.

In one preferred embodiment, the dAK has an amino acid sequence that has at least 65% sequence identity to SEQ ID No 34, preferably at least 70%, more preferably at least 75%, more preferably at least 80%, more preferably at least 85%, more preferably 90%, more preferably at least 95%, more preferably at least 98%.

In another preferred embodiment the dAK has an amino acid sequence that has at least 65% sequence identity to SEQ ID No 35, preferably at least 70%, more preferably at least 75%, more preferably at least 80%, more preferably at least 85%, more preferably 90%, more preferably at least 95%, more preferably at least 98%.

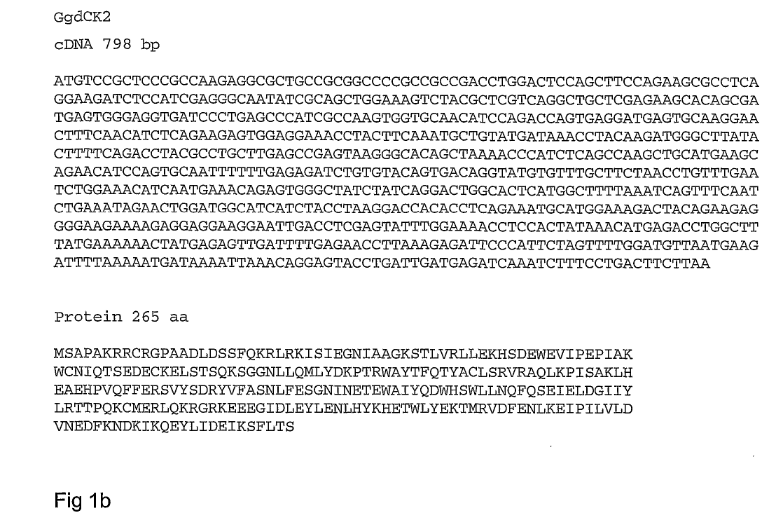

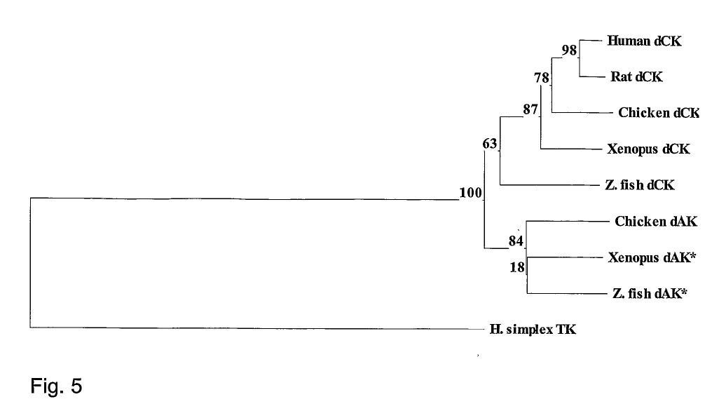

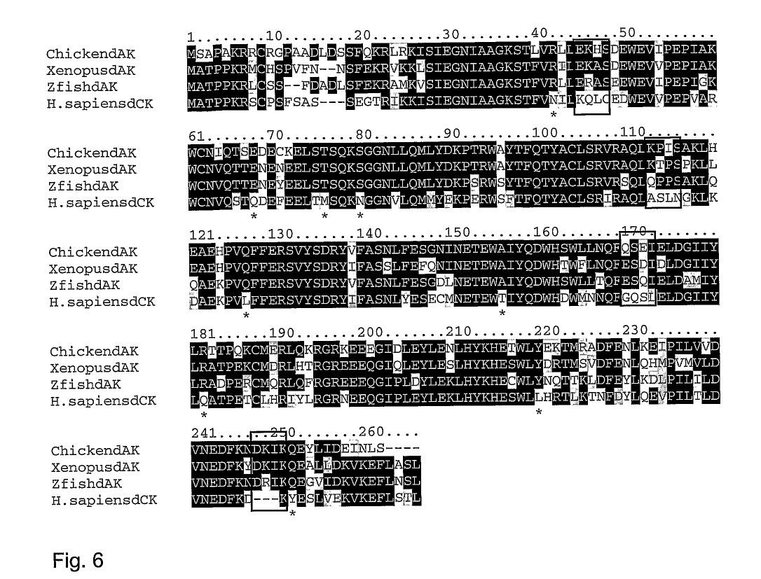

Using the alignment in FIG. 6, it has been possible to identify specific dAK motifs (boxed in the figure). The following parts represent putative dAK specific motifs: region 45-48; 113-116; 170-172 and 248-250. In particular, the region at amino acids 170-172 is believed to be of importance, since the human dCK sequence here is conserved in other dCKs (see FIG. 2). Amino acid positions, believed to distinguish eukaryotic dAKs from eukaryotic dCKs include (numbering according to FIG. 6): 42 (R), 68 (E), 76 (S), 80 (S), 127 (Q), 156 (A), 182 (R), 220 (Y), 252 (Y). Of these the following single amino acids in particular may represent dAK specific amino acids: position 42 (dAKs have R, dCKs have N); position 156 (dAKs have A, dCKs have T). A dAK may also be identified by as such by making a distance tree using Clustal W 1.81 as shown in FIG. 5. An amino acid sequence represents a dAK if it groups together with the chicken dAK (SEQ ID No. 4) in a phylogenetic tree similar to tha one shown in FIG. 5.

Accordingly in a preferred embodiment, a dAK enzyme of the invention has an amino acid sequence which in a multisequence alignment using Clustal W 1.81 forms a phylogenetic sub-group together with Chicken dAK (SEQ ID No. 4) but distinct from GgdCK1 (SEQ ID No. 2), human dCK (SEQ ID No. 31), rat dCK (SEQ ID No. 33), and mouse dCK (SEQ ID No 32.).

The dAK enzymes of the present invention are particularly useful for medical use. This medical use may be for inhibiting a pathogenic agent in a warm-blooded animal. The pathogenic agent may be a virus, a bacterium, or a parasite. It may also be a cancer or tumour cell, an autoreactive immune cell. Preferably the dAK enzymes are formulated for simultaneous, successive or separate administration together with a nucleoside analogue, that can be converted into a cytotoxic drug by said dAK enzyme. Preferred nucleoside analogues include adenosine analogues. Preferred adenosine analogues include but are not limited to Fludarabine and Cladribine. Surprisingly, it has also turned out that ara-G, a guanosine analogue, can be converted into a cytotoxic drug more efficiently by the dAK enzymes of the present invention than by known dCKs or dGKs.

The dAK enzymes may be formulated for administration and may be administered in the same way as described for Chicken dCK enzymes in the present application. The following aspects, described below for Chicken dCKs also apply to the dAKs of the present invention: methods of recombinant producing of kinase enzyme, expression vectors encoding kinase enzymes, pharmaceutical articles comprising kinase enzymes, nucleic acids or host cells, host cells expressing kinase enzymes, gene therapy using kinase enzymes, radionuclide imaging using kinase enzymes. dAK agonists and antagonists can be identified using the methods described for dCKs and dAK enzymes or fragments can be used to generate antibodies using the methods described for generation of antibodies against dCKs.

Gallus gallus dCK Polynucleotides

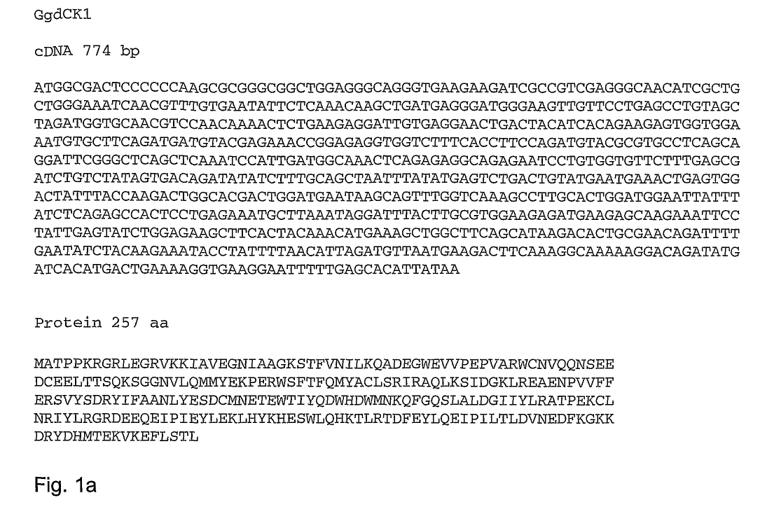

In accordance with one aspect of the present invention, there is provided isolated nucleic acids (polynucleotides), which encode polypeptides having the deduced amino acid sequence of FIG. 1a and b (SEQ ID No. 2 and SEQ ID No. 4).

Polynucleotides encoding polypeptides of the present invention are structurally related to the deoxycytidine kinase family. They contain an open reading frame encoding a protein of 257 amino acid residues (GgdCK1) and 265 amino acid residues (GgdCK2), respectively. The protein exhibits the highest degree of homology to mouse deoxycytidine kinase with 80% identity (213/264) and 89% similarity (237/264) for GgdCK1 and 60% identity (161/265) and 81% similarity (215/265) for GgdCK2. The present case is the first case where two different dCKs have been identified in the same species. Structurally and phylogenetically the two enzymes are classified as dCKs. In the phylogenetic tree shown in FIG. 5, the Gallus gallus kinases group together with other eykaryotic dCKs. GgdCK2 forms its own group together with kinases from Xenopus and Zebrafish. The two kinases grouped together with GgdCK2 have not been characterised yet, but the present inventors believe that they represent further eukaryotic deoxyadenosine kinases. The percent sequence identity among the three deoxyadenosine kinases is approximately 75%.

The polynucleotide of the present invention may be in the form of RNA or in the form of DNA, which DNA includes cDNA, genomic DNA, and synthetic DNA, or PNA or LNA. The DNA may be double-stranded or single-stranded, and if single stranded may be the coding strand or non-coding (anti-sense) strand. The coding sequence which encodes the polypeptide may be identical to the coding sequences shown in FIG. 1 a and b (SEQ ID No. 1 and 3) or may be a different coding sequence which coding sequence, as a result of the redundancy or degeneracy of the genetic code, encodes the same polypeptides as the nucleic acid of FIGS. 1a and b (SEQ ID No. 2 and 4).

The polynucleotides which encode the polypeptides of FIGS. 1a and b (SEQ ID No. 2 and 4) may include: only the coding sequence for the polypeptide; the coding sequence for the polypeptide and additional coding sequence; the coding sequence for the polypeptide (and optionally additional coding sequence) and non-coding sequence, such as introns or non-coding sequence 5′ and/or 3′ of the coding sequence for the polypeptide.

Thus, the term “polynucleotide encoding a polypeptide” encompasses a polynucleotide which includes only coding sequence for the polypeptide as well as a polynucleotide which includes additional coding and/or non-coding sequence.

The present invention further relates to variants of the hereinabove described polynucleotides which encode fragments, analogues, mutants, and derivatives of the polypeptides having the deduced amino acid sequence of FIG. 1 (SEQ ID No.2 and 4).

Thus, the present invention includes polynucleotides encoding the same polypeptides as shown in FIG. 1 (SEQ ID No.2 and 4) as well as variants of such polynucleotides which variants encode a fragment, derivative, mutant, or analogue of the polypeptides of FIG. 1 (SEQ ID NO.2 and 4). Such nucleotide variants include deletion variants, substitution variants and addition or insertion variants.

The polynucleotide may have a coding sequence, which is a naturally occurring allelic variant of the coding sequence shown in FIG. 1 (SEQ ID No.1 and 3). As known in the art, an allelic variant is an alternate form of a polynucleotide sequence, which may have a substitution, deletion or addition of one or more nucleotides, which does not substantially alter the function of the encoded polypeptide.

In a preferred embodiment, the encoded polypeptide has at least 85% sequence identity to SEQ ID No 2, preferably at least 90%, more preferably at least 95%, more preferably at least 98%, more preferably wherein the encoded polypeptide has the amino acid sequence of SEQ ID No.2.

In another preferred embodiment, the polynucleotide has at least 85% sequence identity to the coding sequence of SEQ ID No 1, preferably at least 90%, more preferably at least 95%, more preferably at least 98%, more preferably wherein the polynucleotide has the nucleotide sequence of SEQ ID No 1.

Preferably, the the polynucleotide encoding a dCK is capable of hybridising to a complement of SEQ ID No. 1 under conditions of at least medium stringency, more preferably at least medium/high stringency, more preferably at least high stringency, more preferably very high stringency.

In another preferred embodiment, the encoded polypeptide has at least 65% sequence identity to SEQ ID No 4, preferably at least 70%, more preferably at least 75%, more preferably at least 80%, more preferably at least 85%, more preferably 90%, more preferably at least 95%, more preferably at least 98%, more preferably wherein the encoded polypeptide has the amino acid sequence of SEQ ID No. 4.

In a further preferred embodiment, the polynucleotide has at least 65% sequence identity to the coding sequence of SEQ ID No 3, preferably at least 70%, more preferably at least 75%, more preferably at least 80%, more preferably at least 85%, more preferably at least 90%, more preferably at least 95%, more preferably at least 98%, more preferably wherein the polynucleotide has the nucleotide sequence of SEQ ID No 3.

Preferably, the polynucleotide encoding a dCK is capable of hybridising to a complement of SEQ ID No. 3 under conditions of at least medium stringency, more preferably at least medium/high stringency, more preferably at least high stringency, more preferably very high stringency.

The encoded dCK when compared to human Herpes simplex virus 1 (HSV-TK1) in a eukaryotic cell preferably decreases at least four fold the LD₁₀₀ of at least one nucleoside analogue, preferably wherein said analogue is gemcitabine. More preferably the LD₁₀₀ is decreased at least 10 fold, more preferably at least 50 fold, more preferably at least 100 fold, more preferably at least 250 fold, more preferably at least 1000 fold. In another preferred embodiment said analogue is araG, in particular when the encoded dCK is GgdCK2.

The polynucleotides of the present invention may also have the coding sequence fused in frame to a marker sequence which allows for purification of the polypeptide of the present invention. The marker sequence may be a hexahistidine tag supplied by a pQE-9 vector to provide for purification of the polypeptide fused to the marker in the case of a bacterial host, or, for example the marker sequence may be a hemagglutinin (HA) tag when a mammalian host, e.g. COS-7 cells, is used. The HA tag corresponds to an epitope derived from the influenza hemagglutinin protein (Wilson, I., et al., Cell, 37:767 (184)). In addition, a GST tag such as supplied by the pGEX-2T vector from Pharmacia can be used.

The present invention further relates to polynucleotides which hybridize to the hereinabove-described sequences under conditions of low stringency, preferably medium stringency, more preferably medium/high stringency, more preferably high stringency, more preferably very high stringency. The polynucleotides which hybridize to the hereinabove described polynucleotides in a preferred embodiment encode polypeptides which retain substantially the same biological function or activity as the polypeptide encoded by the cDNA of FIG. 1 (SEQ ID No. 1 or 3).

Gallus gallus dCK Polypeptides

The present invention further relates to GgdCK polypeptides which have the deduced amino acid sequence of FIG. 1 (SEQ ID No. 2 and 4), as well as fragments, analogs and derivatives of such polypeptides.

The terms “fragment,” “derivative” and analog” when referring to the polypeptides of FIG. 1 (SEQ ID No. 2 and 4), means a polypeptide which retains essentially the same biological function or activity as such polypeptide.

The polypeptide of the present invention may be a recombinant polypeptide, a natural polypeptide or a synthetic polypeptide, preferably a recombinant polypeptide.

The fragment, derivative or analog of the polypeptides of FIG. 1 (SEQ ID No.2 and 4) may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the polypeptide is fused with another compound, such as a compound to increase the half-life of the polypeptide (for example, polyethylene glycol), or (iv) one in which the additional amino acids are fused to the polypeptide, such as a leader or secretory sequence or a sequence which is employed for purification of the polypeptide. Such fragments, derivatives and analogs are deemed to be within the scope of those skilled in the art from the teachings herein.

The multiple sequence analysis of FIG. 2 can be used to identify positions in the primary sequence, which can be modified. If a residue is not conserved among the proteins of FIG. 2 it is an indication that the residue can be substituted (preferably with a residue found at the corresponding position in another deoxycytidine kinase) while conserving a deoxycytidine kinase activity. If it is known from other deoxycytidine kinases that particular mutations can be made, it is likely that corresponding mutations can be made to the chicken dCKs of the present invention with the same outcome. Non-limiting example of mutations include the human triple mutant described in the examples. Further examples of mutants are shown herein. The P-loop and the lid region marked in FIG. 2 are believed to be important for the function of the kinases.

In a preferred embodiment, the encoded polypeptide has at least 85% sequence identity to SEQ ID No 2, preferably at least 90%, more preferably at least 95%, more preferably at least 98%, more preferably the encoded polypeptide has the amino acid sequence of SEQ ID No. 2. Gallus gallus dCK1 is capable of activating at least gemcitabine better than Gallus gallus dCK2.

In another preferred embodiment, the encoded polypeptide has at least 65% sequence identity to SEQ ID No 4, preferably at least 70%, more preferably at least 75%, more preferably at least 80%, more preferably at least 85%, more preferably 90%, more preferably at least 95%, more preferably at least 98%, more preferably the encoded polypeptide has the amino acid sequence of SEQ ID No. 4.

The polypeptides and polynucleotides of the present invention are preferably provided in an isolated form, and preferably are purified to homogeneity.

The term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

Mutated GgdCK

Chicken deoxycytidine kinases may be subject to random or site-directed mutagenesis to change the stability and/or the kinetic properties of the enzymes. Mutagenesis followed by an enzymatic assay may also be used to identify residues in the amino acid sequence which can be readily mutated without substantially altering the kinetic properties, and which residues are crucial for the kinetic properties. Methods for performing random or site-directed mutagenesis are well known in the art. Examples of both random and site-directed mutagenesis are described in the examples.

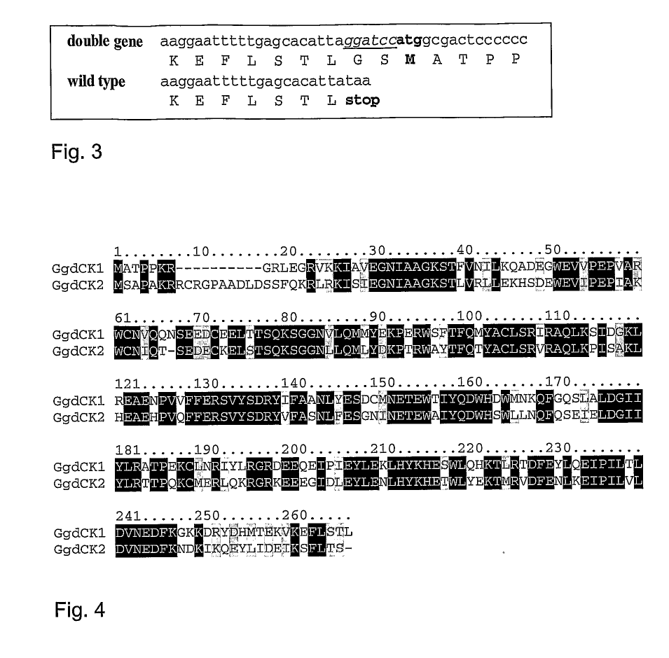

In one embodiment, the mutant is a mutant with respect to the wild-type sequence at one or more of the positions in SEQ ID No. 2 (GgdCK1): 4, 8, 11, 12, 49, 50, 54, 59, 60, 68, 71, 73, 74, 79, 82, 90, 92, 94, 98, 99, 103, 112, 115, 127, 139, 147, 156, 158, 177, 183, 184, 189, 190, 194, 204, 219, 239, and 247. It is expected that the corresponding positions of SEQ ID No. 4 (GgdCK2) can be mutated with similar results. The corresponding positions in SEQ ID No. 4 can be identified using a Clustal X 1.81 alignment such as the one reproduced in FIG. 4.

In a preferred embodiment the mutated Chicken dCK comprises one or more of the following mutations (positions corresponding to SEQ ID No. 2): P4L, E8G, E11G, G12D, A49V, R50G, V54A, E59G, E60G, S68P, S711, G73R, N74S, M79T, K82E, K82R, F90Y, M92V, A94V, R98M, I99V, L103P, E112G, N115S, D127A, D139G, T147S, M156T, K158E, E177K, I183T, Y184C, Y184H, D189G, E190G, I194T, Y204C, F219C, F219L, K239W, and T247I. It is expected that corresponding mutations in SEQ ID No. 4 (GgdCK2) can be performed with similar results.

In a preferred embodiment, the mutated GgdCK comprises mutation(s) selected from the following group of mutations (SEQ ID No. 2 numbering):

F90Y;

E11G/K82E/199V/L103P;

E11G/G12D/A49V/N115S/F219C/T2471;

N74S;

E59G/M79T/Y184C;

E60G/G73R/K82R;

E11G/S71I/M92V/F219L;

P4L/E11G/T147S;

E11G/M79T/D139G/Y184C;

E8G/I194T;

S68P/K158E;

M156T/Y184H/Y204C/K239W;

E112G/I183T;

E11G/E190G;

V54A/E177K;

E11G/R50G;

P4L/D189G;

D127A;

A94V/R98M; and

A94V/R98M/D127A.

All of these mutants when applied to GgdCK1 activate gemcitabine as well as or better than the wildtype GgdCK1.

In a particularly preferred embodiment, the mutated GgdCK comprises mutation(s) selected from the following group of mutations (SEQ ID No. 2 numbering):

E11G/G12D/A49V/N115S/F219C/T247I and N74S. These particular mutations when applied to the GgdCK1 protein results in proteins with an increased selectivity towards gemcitabine compared to wild-type GgdCK1.

Pharmaceutical Articles

In one aspect, the invention provides pharmaceutical articles comprising a source of a Gallus gallus derived dCK or a functional analog thereof and a nucleoside analogue for the simultaneous, separate or successive administration in cancer therapy.

Preferably the nucleoside analogue is a cytidine analogue because the kinases are cytidine kinases. More preferably the nucleoside analogue is gemcitabine, which is activated by both dCK1 and dCK2 from Gallus gallus.

The source of GgdCK may comprise a GgdCK1 or 2 polypeptide, a GgdCK expression vector, a GgdCK host cell, or a GgdCK the packaging cell line as described in the present application.

The nucleoside analogues may be as defined below.

Vectors

The present invention also relates to vectors which include polynucleotides of the present invention, host cells which are genetically engineered with vectors of the invention and the production of polypeptides of the invention by recombinant techniques.

Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the Chicken dCK genes. The culture conditions, such as temperature, pH and the like, are those previously used with the host cell selected for expression, and will be apparent to the ordinarily skilled artisan.

The polynucleotides of the present invention may be employed for producing polypeptides by recombinant techniques. Thus, for example, the polynucleotide may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies.

Suitable expression vectors may be a viral vector derived from Herpes simplex, adenovira, adenoassociated vira, lentivira, retrovira, or vaccinia vira, or from various bacterially produced plasmids, and may be used for in vivo delivery of nucleotide sequences to a whole organism or a target organ, tissue or cell population. Other delivery methods include, but are not limited to, liposome transfection, electroporation, transfection with carrier peptides containing nuclear or other localising signals, and gene delivery via slow-release systems.

Other suitable expression vectors include general purpose mammalian vectors which are also obtained from commercial sources (Invitrogen Inc., Clonetech, Promega, BD Biosecences, etc) and contain selection for Geneticin/neomycin (G418), hygromycin B, puromycin, Zeocin/bleomycin, blasticidin SI, mycophenolic acid or histidinol.

The expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vectors include the following classes of vectors: general eukaryotic expression vectors, vectors for stable and transient expression and epitag vectors as well as their TOPO derivatives for fast cloning of desired inserts (see list below for available vectors).

Ecdysone-Inducible Expression: pIND(SP1) Vector; pINDN5-His Tag Vector Set; pIND(SP1)N5-His Tag Vector Set; EcR Cell Lines; Muristerone A.

Stable Expression: pcDNA3.1/Hygro; pSecTag A, B & C; pcDNA3.1(−)/MycHis A, B & C pcDNA3.1±; pcDNA3.1/Zeo (+) and pcDNA3.1/Zeo (−); pcDNA3.1/His A, B, & C; pRc/CMV2; pZeoSV2 (+) and pZeoSV2 (−); pRc/RSV; pTracer™-CMV; pTracer™-SV40.

Transient Expression: pCDM8; pcDNA1.1; pcDNA1.1/Amp.

Epitag Vectors: pcDNA3.1/MycHis A, B & C; pcDNA3.1N5-His A, B, & C.

Large numbers of suitable vectors and promoters are known to those of skill in the art, and are commercially available. The following vectors are provided by way of example. Bacterial: pQE70, pQE60, pQE-9 (Qiagen), pBS, pD10, phagescript, psiX174, pbluescript SK, pbsks, pNH8A, pNH16a, pNH18A, pNH46A (Stratagene); ptrc99a, pKK2233, pKK233-3, pDR540, pRITS (Pharmacia). Eukaryotic: pWLNEO, pSV2CAT, pOG44, pXTI, pSG (Stratagene) pSVK3, pBPV, pMSG, pSVL (Pharmacia). Mammalian: pCl, pSI (Promega). However, any other plasmid or vector may be used as long as they are replicable and viable in the host.

In a gene therapy approach the dCK of the present invention can be overexpressed in tumour cells by placing the gene coding for said dCK under the control of a strong constitutive or tissue specific promoter, such as the CMV promoter, human UbiC promoter, JeT promoter (U.S. Pat. No. 6,555,674), SV40 promoter, and Elongation Factor 1 alpha promoter (EF1-alpha). Another type of preferred promoters include tissue specific promoters, which preferably encompass promoters that are expressed specifically in cancer cells (e.g. the intermediate filament protein nestin promoter promotes cell-specific expression in neuro-epithelial cells of stem cell or malignant phenotype (Lothian, C. et al., 1999, Identification of both general and region-specific embryonic CNS enhancer elements in the nestin promote, Exp.Cell Res., 248:509-519). Other suitable examples of tissue specific promoters include: PSA prostate specific antigen (prostate cancer); AFP Alpha-Fetoprotein (hepatocellular carcinoma); CEA Carcinoembrionic antigen (epithelial cancers); COX-2 Cyclo-oxygenase 2 (tumour); MUC1 Mucin-like glycoprotein (carcinoma cells); E2F-1 E2F transcription factor 1 (tumour). Human telomerase reverse transcriptase (hTERT), the catalytic subunit of telomerase functions to stabilise telomere length during chromosomal replication. Previous studies have shown that hTERT promoter is highly active in most tumour tissue and immortal cell lines, but inactive in normal somatic cell types.

The DNA sequence in the expression vector is operatively linked to an appropriate expression control sequence(s) (promoter) to direct mRNA synthesis. As representative examples of such promoters, there may be mentioned: LTR or SV40 promoter, the E. coli. lac or trod, the phage lambda PL promoter and other promoters known to control expression of genes in prokaryotic or eukaryotic cells or their viruses.

Promoter regions can be selected from any desired gene using CAT (chloramphenicol transferase) vectors or other vectors with selectable markers. Two appropriate vectors are PKK232-8 and PCM7. Particular named bacterial promoters include lac, lacZ, T3, T7, gpt, lambda PR, PL and trp.

Eukaryotic promoters include v immediate early, HSV thymidine kinase, early and late SV40, LTRs from retrovirus, and mouse metallothionein-I. Selection of the appropriate vector and promoter is well within the level of ordinary skill in the art.

The constructs in host cells can be used in a conventional manner to produce the gene product encoded by the recombinant sequence. Alternatively, the polypeptides of the invention can be synthetically produced by conventional peptide synthesizers.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The expression vector also contains a ribosome binding site for translation initiation and a transcription terminator.

The vector may also include appropriate sequences for amplifying expression.

Proteins can be expressed in mammalian cells, yeast, bacteria, or other cells under the control of appropriate promoters. Cell-free translation systems can also be employed to produce such proteins using RNAs derived from the DNA constructs of the present invention.

Appropriate cloning and expression vectors for use with prokaryotic and eukaryotic hosts are described by Sambrook, et al., Molecular Cloning: A Laboratory Manual, Second Edition, Cold Spring Harbor, N.Y., (1989), the disclosure of which is hereby incorporated by reference.

Transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription.

Examples including the SV40 enhancer on the late side of the replication origin bp 100 to 270, a cytomegalovirus early promoter enhancer, the polyoma enhancer on the late side of the replication origin., and adenovirus enhancers.

Optionally, the heterologous sequence can encode a fusion protein including an N-terminal identification peptide imparting desired characteristics, e.g., stabilization or simplified purification of expressed recombinant product.

As a representative but nonlimiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wis., USA). These pBR322 “backbone” sections are combined with an appropriate promoter and the structural sequence to be expressed.

Host Cells

As representative examples of appropriate hosts, there may be mentioned: bacterial cells, such as E. coli, Bacillus subtilis, Streptomyces, Salmonella typhimurium, Pseudomonas species, Staphylococcus sp.; fungal cells, such as yeast; insect cells such as Drosophilia S2 and Spodoptera Sf9; animal cells such as CHO, COS or Bowes melanoma; adenovirus; plant cells, etc. The selection of an appropriate host is deemed to be within the scope of those skilled in the art from the teachings herein.

In a preferred embodiment the host cell of the invention is a eukaryotic cell, in particular a mammalian cell, a human cell, an oocyte, or a yeast cell. In a more preferred embodiment the host cell of the invention is a human cell, a dog cell, a monkey cell, a rat cell or a mouse cell.

The human cells may be human stem cells or human precursor cells, such as human neuronal stem cells, and human hematopoietic stem cells etc capable of forming tight junctions with cancer cells. These may be regarded as therapeutic cell lines and can be administered to a subject in need thereof. Stem cells have the advantage that they can migrate in the body and form tight junctions with cancer cells. Upon administration of a nucleoside analogue prodrug, this is converted into a cytotoxic drug by the stem cell kinase and the stem cell is killed selectively together with cancer cells. Non-limiting examples of committed precursor cells include hematopoietic cells, which are pluripotent for various blood cells; hepatocyte progenitors, which are pluripotent for bile duct epithelial cells and hepatocytes; and mesenchymal stem cells. Another example is neural restricted cells, which can generate glial cell precursors that progress to oligodendrocytes and astrocytes, and neuronal precursors that progress to neurons.

Migrating cells that are capable of tracking down glioma cells and that have been engineered to deliver a therapeutic molecule represent an ideal solution to the problem of glioma cells invading normal brain tissue. It has been demonstrated that the migratory capacity of neural stem cells (NSCs) is ideally suited to therapy in neurodegenerative disease models that require brain-wide cell replacement and gene expression. It was hypothesized that NSCs may specifically home to sites of disease within the brain. Studies have also yielded the intriguing observation that transplanted NSCs are able to home into a primary tumor mass when injected at a distance from the tumor itself; furthermore, NSCs were observed to distribute themselves throughout the tumor bed, even migrating in juxtaposition to advancing single tumor cells (Dunn & Black, Neurosurgery 2003, 52:1411-1424; Aboody et al, PNAS, 2000, 97:12846-12851). These authors showed that NSCs were capable of tracking infiltrating glioma cells in the brain tissue peripheral to the tumor mass, and “piggy back” single tumor cells to make cell-to-cell-contact.

Engineered NSCs expressing an enzyme that can activate a prodrug can be used to track and destroy advancing glioma cells.

Preferably the kind of stem cell used for this type of therapy originates from the same tissue as the tumour cell or from the same growth layer. Alternatively, the stem cells may originate from bone marrow. The stem cells may be isolated from the patient (e.g. bone marrow stem cells), be engineered to over-express a deoxyribonucleoside kinase and be used in the same patient (autograft). For use in the CNS, where graft-host incompatibility does not constitute a significant problem, the cells may originate from a donor (allograft). The donor approach is preferred for the CNS as this makes it possible to produce large quantities of well-characterised stem cells, which can be stored and are ready for use. It is also contemplated to use xenografts, i.e. stem cells originating from another species, such as other primates or pigs. Cells for xenotransplantation may be engineered to reduce the risk of tissue rejection.

Bone marrow transplantation is more and more adopted as a therapy for a number of malignant and non-malignant haematological diseases, including leukemia, lymphoma, aplastic anemia, thalassemia major and immunodeficiency diseases in general. Since donor marrow contains immunocompetent cells, the graft rejects the host (causing so called graft-versus-host disease, GVHD) in 50-70% of the transplant patients, resulting in generalised inflammatory erythrodema of the skin, gastrointestinal haemorrhage and liver failure. Over 90% of GVHD cases are fatal. Although various treatments are administered to prevent GVHD in bone marrow transplantation there is clear need for safety mechanisms, which can be activated on demand to kill transplanted cells. By incorporating a kinase of the present invention into donor cells prior to transplantation, these cells are rendered susceptible to nucleoside analogues. Nucleoside analogues can be administered in case of GVHD to stop deadly GVHD. This “safety switch” can be refined further by placing the introduced kinase under the control of a strong inducible promoter, e.g. Tet on-off.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE Dextran mediated transfection, or electroporation. (Davis, L., Dibner, M., Battey, I., Basic Methods in Molecular Biology, (1986)).

Recombinant Production of Gallus gallus dCKs

Following transformation or transduction of a suitable host strain and growth of the host strain to an appropriate cell density, the selected promoter is induced by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well know to those skilled in the art. Once example is expression and purification of a GST-tagged dCK as described in the examples. The GST tag may be cleaved from kinase.

Various mammalian cell culture systems can also be employed to express recombinant protein. Examples of mammalian expression systems include the COS-7 lines of monkey kidney fibroblasts, described by Gluzman, Cell, 23:175 (1981), and other cell lines capable of expressing a compatible vector, for example, the C127, 3T3, CHO, HeLa and BHK cell lines.

The chicken dCK polypeptides can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography hydroxylapatite chromatography and lectin chromatography. Protein refolding steps can be used, as necessary, in completing configuration of the protein. Finally, high performance liquid chromatography (HPLC) can be employed for final purification steps.

The polypeptides of the present invention may be a naturally purified product, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated or may be non-glycosylated.

Gene Therapy

The chicken dCK polypeptides and agonists and antagonists which are polypeptides, discussed below, may also be employed in accordance with the present invention by expression of such polypeptides in vivo, which is often referred to as “gene therapy.”

Thus, for example, cells from a patient may be engineered with a polynucleotide (DNA or RNA) encoding a polypeptide ex vivo, with the engineered cells then being provided to a patient to be treated with the polypeptide.

Such methods are well-known in the art. For example, cells may be engineered by procedures known in the art by use of a retroviral particle containing RNA encoding a polypeptide of the present invention. For example, the expression vehicle for engineering cells may be other than a retrovirus, for example, an adenovirus which may be used to engineer cells in vivo after combination with a suitable delivery vehicle. Most preferable is oncolytic adenovirus (replication competent adenovirus) and adenovirus. MV and lentivirus are also preferred for some cancer applications as both types of vectors have been tested in clinical trials.

In U.S. Pat. No. 6,627,442 methods and viruses for efficient transduction of primary hematopoietic cells and hematopoietic stem cells are described.

Similarly, cells may be engineered in vivo for expression of a polypeptide in vivo by, for example, procedures known in the art and described in the present application.

Alternatively and for prolonged delivery of virus particles, a producer cell for producing a retroviral particle containing RNA encoding the polypeptide of the present invention may be administered to a patient for engineering cells in vivo and expression of the polypeptide in vivo.

These and other methods for administering a polypeptide of the present invention by such method should be apparent to those skilled in the art from the teachings of the present invention.

Once the chicken dCK polypeptides are being expressed intracellularly via gene therapy, it may be employed to treat malignancies, e.g., tumors, cancer, leukemias and lymphomas and viral infections, since chiken dCKs catalyze the initial phosphorylation step in the formation of cytotoxic triphosphate derivatives of nucleosides such as gemcitabine, ara-C, 2 fluoro-9-S-D-arabinofuranosyladenine and dideoxycytidine.

The kinases of the invention may be used as a “safety switch” in donor cells prior to transplantation into the host to make it possible to selectively kill the transplanted cells in the case of GVHD or in other cases, where there is a need to remove transplanted cells.

Chicken dCK polypeptides may also be employed to maintain normal deoxyribonucleotide pools and therefore ensure correct DNA synthesis.

Prodrugs and Nucleoside Analogues

The present invention in several aspects relates to the simultaneous, separate or successive use of the dCKs of the invention and a prodrug, which can be activated by a dCK of the invention.

In a preferred embodiment the prodrug is a nucleoside analogue. On a functional level, a nucleoside analogue is a compound with a molecular weight less than 1000 Daltons, which is substantially non-toxic to human cells, which can be phosphorylated by a deoxyribonucleoside kinase to mono, di, and tri phosphate, the triphosphate of which is toxic to dividing human cells.

According to the methods of the present invention at least two or more different nucleoside analogues, such as at least 3 nucleoside analogues, for example at least 4 nucleoside analogues, such as at least 5 nucleoside analogues may be administered to the same subject.

Numerous nucleoside analogs exist that can be converted into a toxic product including a large group described in US 20040002596.

In a preferred embodiment the nucleoside analogue include a compound selected from the group consisting of aciclovir (9-[2-hydroxy-ethoxy]-methyl-guanosine), buciclovir, famciclovir, ganciclovir (9-[2-hydroxy-1-(hydroxymethyl)ethoxyl-methyl]-guanosine), penciclovir, valciclovir, trifluorothymidine, AZT (3′-azido-3′-thymidine), AIU (5′-iodo-5′-amino-2′,5′-dideoxyuridine), ara-A (adenosine-arabinoside; Vivarabine), ara-C (cytidine-arabinoside), ara-G (9-beta-D-arabinofuranosylguanine), ara-T, 1-beta-D-arabinofuranosyl thymine, 5-ethyl-2′-deoxyuridine, 5-iodo-5′-amino-2,5′-dideoxyuridine, 1-[2-deoxy-2-fluoro-beta-D-arabino furanosyl]-5-iodouracil, idoxuridine (5-iodo-2′deoxyuridine), fludarabine (2-Fluoroadenine 9-beta-D-Arabinofuranoside), gencitabine, 3′-deoxyadenosine (3-dA), 2′,3′-dideoxyinosine (ddl), 2′,3′-dideoxycytidine (ddC), 2′,3′-dideoxythymidine (ddT), 2′,3′-dideoxyadenosine (ddA), 2′,3′-dideoxyguanosine (ddG), 2-chloro-2′-deoxyadenosine (2CdA), 5-fluorodeoxyuridine, BVaraU ((E)-5-(2-bromovinyl)-1-beta-D-arabinofuranosyluracil), BVDU (5-bromovinyl-deoxyuridine), FIAU (1-(2-deoxy-2-fluoro-beta-D-arabinofuranosyl)-5-iodouracil), 3TC (2′-deoxy-3′-thiacytidine), dFdC gemcitabine (2′,2′-difluorodeoxycytidine), dFdG (2′,2′-difluorodeoxyguanosine), 5-fluorodeoxyuridine (FdUrd), d4T (2′,3′didehydro-3′-deoxythymidine), ara-M (6-methoxy purinearabinonucleoside), ludR (5-Jodo-2′deoxyuridine), CaFdA (2-chloro-2-ara-fluoro-deoxyadenosine), ara-U (1-beta-D-arabinofuranosyluracil), FBVAU (E)-5-(2-bromovinyl)-1-(2-deoxy-2-fluoro-beta-D-arabinofuranosyl)uracil, FMAU 1-(2-deoxzy-2-fluoro-beta-D-arabinofuranosyl)-5-methyluracil, FLT 3′-fluoro-2′-deoxythymidine, 5-Br-dUrd 5-bromodeoxyuridine, 5-Cl-dUrd 5-chlorodeoxyuridine, dFdU 2′,2-difluorodeoxyuridine, (−)Carbovir (C-D4G), 2,6-Diamino-ddP (ddDAPR; DAPDDR; 2,6-Diamino-2′,3′-dideoxypurine-9-ribofuranoside), 9-(2′-Azido-2′,3′-dideoxy-p-D-erythropento-furanosyl)adenine (2′-Azido-2′,3′-dideoxyadenosine; 2′-N3ddA), 2° FddT (2′-Fluoro-2′,3′-dideoxy-β-D-erythro-pentofuranosyl)thymine), 2′-N3ddA(β-D-threo) (9-(2′-Azido-2′,3′-dideoxy-β-D-threopentofuranosyl)adenine), 3-(3-Oxo-1-propenyl)AZT (3-(3-Oxo-1-propenyl)-3′-azido-3′-deoxythymidine), 3′-Az-5-Cl-ddC (3′-Azido-2′,3′-dideoxy-5-chlorocytidine), 3′-N3-3′-dT (3′-Azido-3′-deoxy-6-azathymidine), 3′-F-4-Thio-ddT (2′,3′-Dideoxy-3′-fluoro-4-thiothymidine), 3′-F-5-Cl-ddC (2′,3′-Dideoxy-3′-fluoro-5-chlorocytidine), 3′-FddA (B-D-Erythro) (9-(3′-Fluoro-2′,3′-dideoxy-B-D-erythropentafuranosyl)adenine), Uravidine (3′-Azido-2′,3′-dideoxyuridine; AzdU), 3′-FddC (3′-Fluoro-2′,3′-dideoxycytidine), 3′-F-ddDAPR (2,6-Diaminopurine-3′-fluoro-2′,3′-dideoxyriboside), 3′-FddG (3′-Fluoro-2′,3′-dideoxyguanosine), 3′-FddU (3′-Fluoro-2′,3′-dideoxyuridine), 3′-Hydroxvmethyl-ddC (2′,3′-Dideoxy-3′-hydroxymethyl cytidine; BEA-005), 3′-N3-5-CF3-ddU (3′-Azido-2′,3′-dideoxy-5-trifluoromethyluridine), 3′-N3-5-Cyanomethyl-ddU (3′-Azido-2′,3′-dideoxy-5-[(cyanomethyl)oxy]uridine), 3′-N3-5-F-ddC (3′-Azido-2′,3′-dideoxy-5-fluorocytidine), 3′-N3-5-Me-ddC(CS-92; 3′-Azido-2′,3′-dideoxy-5-methylcytidine), 3′-N3-5-NH2-ddU (3′-Azido-2′,3′-dideoxy-5-aminouridine), 3′-N3-5-NHMe-ddU (3′-Azido-2′,3′-dideoxy-5-methyaminouridine), 3′-N3-5-NMe2-ddU (3′-Azido-2′,3′-dideoxy-5-dimethylaminouridine), 3′-N3-5-OH-ddU (3′-Azido-2′,3′-dideoxy-5-hydroxyuridine), 3′-N3-5-SCN-ddU (3′-Azido-2′,3′-dideoxy-5-thiocyanatouridine), 3′-N3-ddA (9-(3′-Azido-2′,3′-dideoxy-B-D-erythropentafuranosyl)adenine), 3′-N3-ddC (CS-91; 3′-Azido-2′,3′-dideoxycytidine), 3′-N3ddG (AZG; 3′-Azido-2′,3′-dideoxyguanosine), 3′-N3-N4-5-diMe-ddC (3′-Azido-2′,3′-dideoxy-N4-5-dimethylcytidine), 3′-N3-N4-OH-5-Me-ddC (3′-Azido-2′,3′-dideoxy-N4-OH-5-methylcytidine), 4′-Az-3′-dT (4′-Azido-3′-deoxythymidine), 4′-Az-5CldU (4′-Azido-5-chloro-2′-deoxyuridine), 4′-AzdA (4′-Azido-2′-deoxyadenosine), 4′-AzdC (4′-Azido-2′-deoxycytidine), 4′-AzdG (4′-Azido-2′-deoxyguanosine), 4′-Azdl (4′-Azido-2′-deoxyinosine), 4′-AzdU (4′-Azido-2′-deoxyuridine), 4′-Azidothymidine (4′-Azido-2′-deoxy-.beta.-D-erythro-pentofuranosyl-5-methyl-2,4-dioxopyrimidine), 4′-CN-T (4′-Cyanothymidine), 5-Et-ddC (2′,3′-Dideoxy-5-ethylcytidine), 5-F-ddC (5-Fluoro-2′,3′-dideoxycytidine), 6Cl-ddP (D2ClP; 6-Chloro-ddP; CPDDR; 6-Chloro-9-(2,3-dideoxy-.beta.-D-glyceropentofuranosyl)-9H-purine), 935U83 (2′,3′-Dideoxy-3′-fluoro-5-chlorouridine; 5-Chloro-2′,3′-dideoxy-3′-fluorouridine; FddClU; Raluridine), AZddBrU (3′-N3-5-Br-ddU; 3′-Azido-2′,3′-dideoxy-5-bromouridine), AzddClU; AzddClUrd (3′-Azido-5-chloro-2′,3′-dideoxyuridine), AZddEtU (3′-N3-5-EtddU; CS-85; 3′-Azido-2′,3′-dideoxy-5-ethyluridine), AZddFU (3′-Azido-2′,3′-dideoxy-5-fluorouridine), AZddlU (3′-N3-5-I-ddU; 3′-Azido-2′,3′-dideoxy-5-iodouridine), AZT-2,5′-anhydro (2,5′-Anhydro-3′-azido-3′-deoxythymidine), AZT-α-L (α-L-AZT), AZU-2,5′-anhydro (2,5′-Anhydro-3′-azido-2′,3′-dideoxyuridine), C-analog of 3′-N3-ddU (3′-Azido-2′,3′-dideoxy-5-aza-6-deazauridine), D2SMeP (9-(2,3-Dideoxy-β-D-ribofuranosyl)-6-(methylthio)purine), D4A (2′,3′-Dideoxydidehydroadenosine), D4C (2′,3′-Didehydro-3′-deoxycytidine), D4DAP (2,6-Diaminopurine-2′,3′-dideoxydidehydroriboside; ddeDAPR), D4FC (D-D4FC; 2′,3′-Didehydro-2′,3′-dideoxy-5-fluorocytidine), D4G (2′,3′-Didehydro-2′,3′-dideoxyguanosine), DMAPDDR (N-6-dimethyl ddA; 6-Dimethylaminopurine-2′,3′-dideoxyriboside), dOTC (−) ((−)-2′-Deoxy-3′-oxa-4′-thiocytidine), dOTC (+) ((+)-2′-Deoxy-3′-oxa-4′-thiocytidine), dOTFC (−) ((−)-2′-Deoxy-3′-oxa-4′-thio-5-fluorocytidine), dOTFC (+) ((+)-2′-Deoxy-3′-oxa-4′-thio-5-fluorocytidine), DXG ((−)-β-Dioxolane-G), DXC-α-L-(α-L-Dioxalane-C), FddBrU (2′,3′-Dideoxy-3′-fluoro-5-bromouridine), FddlU (3′-Fluoro-2′,3′-dideoxy-5-iodouridine), FddT (Alovudine; 3′-FddT; FddThD; 3′-FLT; FLT), FTC (Emtricitabine; Coviracil; (−)-FTC; (−)-2′,3′-Dideoxy-5-fluoro-3′-thiacytidine), FTC-α-L- (α-L-FTC), L-D4A (L-2′,3′-Didehydro-2′,3′-dideoxyadenosine), L-D4FC (L-2′,3′-Didehydro-2′,3′-dideoxy-5-fluorocytidine), L-D4I (L-2′,3′-Didehydro-2′,3′-dideoxyinosine), L-D4G (L-2′,3′-Didehydro-2′,3′-deoxyguanosine), L-FddC (β-L-5F-ddC), Lodenosine (F-ddA; 2′-FddA (B-D-threo); 2′-F-dd-ara-A; 9-(2′-Fluoro-2′,3′-dideoxy-B-D-threopentafuranosyl)adenine), MeAZddIsoC (5-Methyl-3′-azido-2′,3′-dideoxyisocytidine), N6-Et-ddA (N-Ethyl-2′,3′-dideoxyadenosine), N-6-methyl ddA (N6-Methyl-2′,3′-dideoxyadenosine) or RO31-6840 (1-(2′,3′-Dideoxy-2′-fluoro-β-D-threo-pentofuranosyl)cytosine).

Preferred examples of cytidine, guanosine and adenosine analogs include dFdC gemcitabine (2′,2′-difluorodeoxycytidine), 2-chloro-2′-deoxyadenosine (2CdA), CaFdA (2-chloro-2-ara-fluoro-deoxyadenosine), fludarabine (2-Fluoroadenine 9-beta-D-Arabinofuranoside), 2′,3′-dideoxycytidine (ddC), 2′,3′-dideoxyadenosine (ddA), 2′,3′-dideoxyguanosine (ddG), ara-A (adenosine-arabinoside; Vivarabine), ara-C (cytidine-arabinoside), ara-G (9-beta-D-arabinofuranosylguanine), aciclovir (9-[2-hydroxy-ethoxy]-methyl-guanosine), buciclovir, famciclovir, ganciclovir (9-[2-hydroxy-1-(hydroxymethyl)ethoxyl-methyl]-guanosine), penciclovir, valciclovir, 3TC (2′-deoxy-3′-thiacytidine), dFdG (2′,2′-difluorodeoxyguanosine), 2,6-Diamino-ddP (ddDAPR; DAPDDR; 2,6-Diamino-2′,3′-dideoxypurine-9-ribofuranoside), 9-(2′-Azido-2′,3′-dideoxy-β-D-erythropentofuranosyl)adenine (2′-Azido-2′,3′-dideoxyadenosine; 2′-N3ddA), 2′-N3ddA(β-D-threo) (9-(2′-Azido-2′,3′-dideoxy-β-D-threopentofuranosyl)adenine), 3′-Az-5-Cl-ddC (3′-Azido-2′,3′-dideoxy-5-chlorocytidine), 3′-F-5-Cl-ddC (2′,3′-Dideoxy-3′-fluoro-5-chlorocytidine), 3′-FddA (B-D-Erythro) (9-(3′-Fluoro-2′,3′-dideoxy-B-D-erythropentafuranosyl)adenine), 3′-FddC (3′-Fluoro-2′,3′-dideoxycytidine), 3′-F-ddDAPR (2,6-Diaminopurine-3′-fluoro-2′,3′-dideoxyriboside), 3′-FddG (3′-Fluoro-2′,3′-dideoxyguanosine), 3′-Hydroxymethyl-ddC (2′,3′-Dideoxy-3′-hydroxymethyl cytidine; BEA-005), 3′-N3-5-F-ddC (3′-Azido-2′,3′-dideoxy-5-fluorocytidine), 3′-N3-5-Me-ddC (CS-92; 3′-Azido-2′,3′-dideoxy-5-methylcytidine), 3′-N3-ddA (9-(3′-Azido-2′,3′-dideoxy-B-D-erythropentafuranosyl)adenine), 3′-N3-ddC (CS-91; 3′-Azido-2′,3′-dideoxycytidine), 3′-N3ddG (AZG; 3′-Azido-2′,3′-dideoxyguanosine), 3′-N3-N4-5-diMe-ddC (3′-Azido-2′,3′-dideoxy-N4-5-dimethylcytidine), 3′-N3-N4-OH-5-Me-ddC (3′-Azido-2′,3′-dideoxy-N4-OH-5-methylcytidine), 4′-AzdA (4′-Azido-2′-deoxyadenosine), 4′-AzdC (4′-Azido-2′-deoxycytidine), 4′-AzdG (4′-Azido-2′-deoxyguanosine), 5-Et-ddC (2′,3′-Dideoxy-5-ethylcytidine), 5-F-ddC (5-Fluoro-2′,3′-dideoxycytidine), 6Cl-ddP (D2ClP; 6-Chloro-ddP; CPDDR; 6-Chloro-9-(2,3-dideoxy-.beta.-D-glyceropentofuranosyl)-9H-purine), D2SMeP (9-(2,3-Dideoxy-β-D-ribofuranosyl)-6-(methylthio)purine), D4A (2′,3′-Dideoxydidehydroadenosine), D4C (2′,3′-Didehydro-3′-deoxycytidine), D4DAP (2,6-Diaminopurine-2′,3′-dideoxydidehydroriboside; ddeDAPR), D4FC (D-D4FC; 2′,3′-Didehydro-2′,3′-dideoxy-5-fluorocytidine), D4G (2′,3′-Didehydro-2′,3′-dideoxyguanosine), DMAPDDR (N-6-dimethyl ddA; 6-Dimethylaminopurine-2′,3′-dideoxyriboside), dOTC (−) ((−)-2′-Deoxy-3′-oxa-4′-thiocytidine), dOTC (+) ((+)-2′-Deoxy-3′-oxa-4′-thiocytidine), dOTFC (−) ((−)-2′-Deoxy-3′-oxa-4′-thio-5-fluorocytidine), dOTFC (+) ((+)-2′-Deoxy-3′-oxa-4′-thio-5-fluorocytidine), DXG ((−)-β-Dioxolane-G), DXC-α-L-(α-L-Dioxalane-C), FTC (Emtricitabine; Coviracil; (−)-FTC; (−)-2′,3′-Dideoxy-5-fluoro-3′-thiacytidine), FTC-α-L- (α-L-FTC), L-D4A (L-2′,3′-Didehydro-2′,3′-dideoxyadenosine), L-D4FC (L-2′,3′-Didehydro-2′,3′-dideoxy-5-fluorocytidine), L-D4I (L-2′,3′-Didehydro-2′,3′-dideoxyinosine), L-D4G (L-2′,3′-Didehydro-2′,3′-deoxyguanosine), L-FddC (β-L-5F-ddC), Lodenosine (F-ddA; 2′-FddA (B-D-threo); 2′-F-dd-ara-A; 9-(2′-Fluoro-2′,3′-dideoxy-B-D-threopentafuranosyl)adenine), MeAZddIsoC (5-Methyl-3′-azido-2′,3′-dideoxyisocytidine), N6-Et-ddA (N-Ethyl-2′,3′-dideoxyadenosine), N-6-methyl ddA (N6-Methyl-2′,3′-dideoxyadenosine) or RO31-6840 (1-(2′,3′-Dideoxy-2′-fluoro-β-D-threo-pentofuranosyl)cytosine).

Preferably, the nucleoside analogue is a cytidine analogue. The kinases of the present invention are cytidine kinases and are expected to act on cytidine analogues. Furthermore it has been shown that the dCKs of the present invention are capable of phorphorylating gemcitabine, which is a cytidine analog.

Several nucleoside analogues have been approved by the FDA as drugs and there is ample knowledge concerning the dosages required to obtain therapeutic efficacy for the approved drugs D4T, ddC, dFdC, AZT, ACV, 3TC, ddA, fludarabine, Cladribine, araC, gemcitabine, Clofarabine, Nelarabine (araG) and Ribarivin.

Particularly preferred combinations of nucleoside analogues and kinase according to the present invention are GgdCK1 with gemcitabine and GgdCK2 (GgdAK) with araG.

Other Uses

In accordance with yet a further aspect of the present invention, there is provided a process for utilizing such polypeptides, or polynucleotides encoding such polypeptides, for in vitro purposes related to scientific research, synthesis of DNA and manufacture of DNA vectors and to design therapeutics to treat human disease.

Fragments of the full length of the chicken dCK genes may be used as a hybridization probe for a cDNA library to isolate the full length dCK genes and to isolate other genes which have a high sequence similarity to the chicken dCK genes or similar biological activity. Probes of this type generally have at least 20 bases. Preferably, however, the probes have at least 30 bases and generally do not exceed 50 bases, although they may have a greater number of bases. The probe may also be used to identify a cDNA clone corresponding to a full length transcript and a genomic clone or clones that contain the complete chicken dCK genes including regulatory and promotor regions, exons, and introns. An example of a screen comprises isolating the coding region of the chicken dCK genes by using the known DNA sequence to synthesize an oligonucleotide probe. Labeled oligonucleotides having a sequence complementary to that of the gene of the present invention are used to screen a cDNA library, genomic DNA or mRNA to determine which members of the library the probe hybridizes to.

Chicken dCK Agonists and Antagonists

Chicken dCK polypeptides may also be employed in a method of screening compounds to identify those which enhance (agonists) or block (antagonists) the phosphorylation activity of dCK. An example of such a method comprises isolating dCK from cells or membrane preparations which express dCK, preparing a reaction mixture, dCK enzyme and the compound to be screened. The reaction mixture is then incubated at elevated temperatures and the deoxycytidine monophosphates formed are detected by the DE-81 disk method (Cheng, Y. C. et al., Biochem. Bioohvs. Acta, 481:481-492 (1977)). The dCK activity can be calculated and expressed as pmol of dCMP/min/pg of protein. The ability of the compound to enhance or block the dCK activity as compared to standard activity in the absence of the compound can then be measured.

Chicken dCKs are produced and function intra-cellularly, therefore, any antagonists must be intra-cellular. Potential antagonists to dCK include antibodies which are produced intra-cellularly. For example, an antibody identified as antagonizing dCK may be produced intra-cellularly as a single chain antibody by procedures known in the art, such as transforming the appropriate cells with DNA encoding the single chain antibody to prevent the function of dCK. Due to the similarity between human and chicken dCKs it is expected that some antibodies raised against chicken dCKs have cross reactivity to human dCKs.

A potential dCK antagonist also includes an antisense construct prepared using antisense technology. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the polypeptides of the present invention, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al, Science, 241:456 (1988); and Dervan et al., Science, 251: 1360 (1991)), thereby preventing transcription and the production of hdCK. The anti sense RNA oligonucleotide hybridizes to the mRNA in vivo and blocks translation of the mRNA molecule into the hdCK polypeptides (antisense—Okano, J. Neurochem., 56:560 (1991); Oligodeoxynucleotides as Antisense Inhibitors of Gene Expression, CRC Press, Boca Raton, Fla. (1988)). The oligonucleotides described above can also be delivered to cells such that the antisense RNA or DNA may be expressed in vivo to inhibit production of dCK.

Potential dCK antagonists also include small molecules, which are able to pass through the cell membrane, and bind to and occupy the catalytic site of the polypeptide thereby making the catalytic site inaccessible to substrate such that normal biological activity is prevented. Examples of small molecules include but are not limited to small peptides or peptide-like molecules.

The antagonist may be employed to treat immunodeficiency diseases, since dCK catalyzes a critical step in the synthesis of dATP or dGTP whose accumulation confers cytotoxicity on the T-cell precursors in these disorders.

Accordingly, inhibition of the dCK function can eliminate these disorders. The antagonists may be employed in a composition with a pharmaceutically acceptable carrier, e.g., as hereinafter described.

The small molecule agonists and antagonists of the present invention may be employed in combination with a suitable pharmaceutical carrier. Such compositions comprise a therapeutically effective amount of the polypeptide, and a pharmaceutically acceptable carrier or excipient. Such a carrier includes but is not limited to saline, buffered saline, dextrose, water, glycerol, ethanol, and combinations thereof. The formulation should suit the mode of administration.

Pharmaceutical Compositions

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the pharmaceutical compositions may be employed in conjunction with other therapeutic compounds.

The pharmaceutical compositions may be administered in a convenient manner such as by the oral, topical, intravenous, intraperitoneal, intramuscular, subcutaneous, intranasal or intradermal routes. The pharmaceutical compositions are administered in an amount which is effective for treating and/or prophylaxis of the specific indication.

Guidance to the dosage of dCK protein, dCK virus and nucleoside analogs can be found in the numerous publications describing clinical trials with HSV-TK1 suicide gene therapy. Thymidine kinases, in particular human HSV-TK1 have been used extensively as suicide gene therapy for the treatment of various types of cancer in combination with various nucleoside analogues. Eg. [Klatzmann D, Valery C A, Bensimon G, Marro B, Boyer O, Mokhtari K, Diquet B, Salzmann J L, Philippon J. A phase I/II study of herpes simplex virus type 1 thymidine kinase “suicide” gene therapy for recurrent glioblastoma. Study Group on Gene Therapy for Glioblastoma. Hum Gene Ther. Nov. 20, 1998;9(17):2595-604.1; [Klatzmann D, Cherin P, Bensimon G, Boyer O, Coutellier A, Charlotte F, Boccaccio C, Salzmann J L, Herson S. A phase I/II dose-escalation study of herpes simplex virus type 1 thymidine kinase “suicide” gene therapy for metastatic melanoma. Study Group on Gene Therapy of Metastatic Melanoma. Hum Gene Ther. November 20, 1998;9(17):2585-94.]; [Freytag S O, Stricker H, Pegg J, Paielli D, Pradhan D G, Peabody J, DePeralta-Venturina M, Xia X, Brown S, Lu M, Kim J H. Phase I study of replication-competent adenovirus-mediated double-suicide gene therapy in combination with conventional-dose three-dimensional conformal radiation therapy for the treatment of newly diagnosed, intermediate- to high-risk prostate cancer. Cancer Res. Nov. 1, 2003;63(21):7497-506.]; [Freytag S O, Khil M, Stricker H, Peabody J, Menon M, DePeralta-Venturina M, Nafziger D, Pegg J, Paielli D, Brown S, Barton K, Lu M, Aguilar-Cordova E, Kim J H. Phase I study of replication-competent adenovirus-mediated double suicide gene therapy for the treatment of locally recurrent prostate cancer. Cancer Res. Sep. 1, 2002;62(17):4968-76.]; [Sung M W, Yeh H C, Thung S N, Schwartz M E, Mandeli J P, Chen S H, Woo S L. Intratumoral adenovirus-mediated suicide gene transfer for hepatic metastases from colorectal adenocarcinoma: results of a phase I clinical trial. Mol Ther. September 2001;4(3):182-91.]; [Packer R J, Raffel C, Villablanca J G, Tonn J C, Burdach S E, Burger K, LaFond D, McComb J G, Cogen P H, Vezina G, Kapcala L P. Treatment of progressive or recurrent pediatric malignant supratentorial brain tumors with herpes simplex virus thymidine kinase gene vector-producer cells followed by intravenous ganciclovir administration. J Neurosurg. February 2000;92(2):249-54.].

HSV-TK has been used for treating the following types of cancer, which are amenable to suicide gene therapy according to the present invention. Bladder cancer, Sutton et al 1997, Urology, 49:173-180; Neuroblastoma, Bi, X and Zhang, J-Z. Pediadtr. Surg. Int., 19:400-405, 2003; Glioblastoma, Germano I. M et al. J. Neurooncol., 65:279-289, 2003; Esophageal cancer, Matsubara, H. and Ochiai, Nippon Rinsho. September 2000;58(9):1935-43.; Tongue cancer, Wang, J. H. et al. Chin J. Dent. Res. Dec. 3, 2000(4): 44-48; Hepatocellular carcinoma, Gerolami, R. et al. J. Hepatol. 291-297, 2004; Lung cancer, Kurdow, R. et al. Ann. Thorac. Surg. March 2002; 73(3):905-910; Malignant melanoma, Yamamoto, S. et al. Cancer Gene Therapy, 10:179-186, 2003; Ovarian cancer, Barnes, M. N. and Pustilnik, T. B. Curr. Opin. Obstet Gynecol., 13:47-51, 2001; Prostate cancer. Kubo, H. et al. Human Gene Therapy., 14:227-241, 2003; Renal cell carcinoma, Pulkkanen, K. J. Cancer Gene Therapy, 9:908-916, 2002.

Adaptation of the dosages described in the above identified publications to the dCKs described in the present application are within the capabilities of the preson skilled in the art. Both GgdCK1 and GgdCK2 (GgdAK) of the present invention has better kinetic properties in terms of activation of prodrugs compared to HSK-TK and therefore offer a better alternative to HSV-TK suicide gene therapy.

Antibodies

The polypeptides, their fragments or other derivatives, or analogs thereof, or cells expressing them can be used as an immunogen to produce antibodies thereto. These antibodies can be, for example, polyclonal or monoclonal antibodies.

The present invention also includes chimeric, single chain, and humanized antibodies, as well as F_{ab} fragments, or the product of a F_{ab} expression library. Various procedures known in the art may be used for the production of such antibodies and fragments.

Antibodies generated against the polypeptides corresponding to a sequence of the present invention can be obtained by direct injection of the polypeptides into an animal or by administering the polypeptides to an animal, preferably a nonhuman. The antibody so obtained will then bind the polypeptides itself. In this manner, even a sequence encoding only a fragment of the polypeptides can be used to generate antibodies binding the whole native polypeptides. Such antibodies can then be used to isolate the polypeptide from tissue expressing that polypeptide.

For preparation of monoclonal antibodies, any technique which provides antibodies produced by continuous cell line cultures can be used. Examples include the hybridoma technique (Kohler and Milstein, 1975, Nature, 256:495-497), the trioma technique, the human B-cell hybridoma technique (Kozbor et al., 1983, Immunology Today 4:72), and the EBVhybridoma technique to produce human monoclonal antibodies (Cole, et al., 1985, in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to immunogenic polypeptide products of this invention. Also, transgenic mice may be used to express humanized antibodies to immunogenic polypeptide products of this invention.

Imaging

Suicide gene therapy, i.e. transfection of a so-called suicide gene that sensitizes target cells towards a prodrug, offers an attractive approach for treating malignant tumors. For the development of effective clinical suicide gene therapy protocols, a non-invasive method to assay the extent, the kinetics and the spatial distribution of transgene expression is essential. Such imaging methods allow investigators and physicians to assess the efficiency of experimental and therapeutic gene transfection protocols and would enable early prognosis of therapy outcome.

Radionuclide imaging techniques like single photon emission computed tomography (SPECT) and positron emission tomography (PET), which can non-invasively visualize and quantify metabolic processes in vivo, are being evaluated for repetitive monitoring of transgene expression in living animals and humans. Transgene expression can be monitored directly by imaging the expression of the therapeutic gene itself, or indirectly using a reporter gene that is coupled to the therapeutic gene. Various radiopharmaceuticals have been developed and are now being evaluated for imaging of transgene expression.

Therefore, in another aspect, the invention provides a method of non-invasive nuclear imaging of transgene expression of a chicken deoxycytidine kinase enzyme of the invention in a cell or subject, which method comprises the steps of
(i) transfecting or transducing said cell or subject with a polynucleotide sequence encoding a deoxycytidine kinase enzyme of the invention, which enzyme promotes the conversion of a substrate into a substrate-monophosphate;
(ii) delivering said substrate to said cell or subject; and
(iii) non-invasively monitoring the change to said prodrug in said cell or subject.

In a preferred embodiment the monitoring carried out in step (iii) is performed by Single Photon Emission Computed Tomography (SPECT), by Positron Emission Tomography (PET), by Magnetic Resonance Spectroscopy (MRS), by Magnetic Resonance Imaging (MRI), or by Computed Axial X-ray Tomography (CAT), or a combination thereof.

In a more preferred embodiment the substrate is a labelled nucleoside analogue selected from those listed above. The labelled nucleoside analogue preferably contains at least one radionuclide as a label. Positron emitting radionuclides are all candidates for usage. In the context of this invention the radionuclide is preferably selected from ²H (deuterium), ³H (tritium), ¹¹C, ¹³C, ¹⁴C, ¹⁵O, ¹³N, ¹²³I, ¹²⁵I, ¹³¹I, ¹⁸F and ^{99m}Tc.

An example of commercially available labelling agents, which can be used in the preparation of the labelled nucleoside analogue is [¹¹C]O₂, ¹⁸F, and Nal with different isotopes of Iodine. In particular [¹¹C]O₂ may be converted to a [¹¹C]-methylating agent, such as [¹¹C]H₃1 or [¹¹C]-methyl triflate.

### EXAMPLES

The invention is further illustrated with reference to the following examples, which are not intended to be in any way limiting to the scope of the invention as claimed.

### Example 1

### Identification and Determination of the Sequence of Chicken dCK1 and dCK2

This example describes how the genes encoding the chicken deoxycytidine kinases of the invention were identified, and how vectors to express these kinases were constructed.

As shown in example 3, substantive deoxycytidine kinase activity was found in crude extracts of chicken cells. This led the present inventors to search the expressed sequence tag library of the GeneBank database at the National Institute for Biotechnology Information (http://www.ncbi.nim.nih.gov/) was with the Translated BLAST search Tool (Protein query—Translated db, TBLASTN) to identify Chicken cDNA clones that encode enzymes similar to human dCK enzyme (ACCN P27707). As eukaryots are known to contain only one deoxycytidine kinase, it was expected to identify only one chicken dCK. Several putative EST sequences were determined. Two different EST clones were obtained from Delaware Biotechnology Institute, University of Delaware and plasmids comprising the expressed sequence tag were fully sequenced. Surprisingly, the EST clones encoded two different, and yet functional deoxycytidine kinases. This thus represents the first example ever of a eukaryotic species with more than one deoxycytidine kinase. On closer examination of the kinetics of the purified enzymes, it turned out that one of the genes encoded what looked like an second deoxycytidine kinase, but which is the first known example of a deoxyadenosine kinase from a eukaryotic species.

The DNA sequence determination of clone pgpln.pk001.f17 revealed an ORF of 774 bp (SEQ.ID.NO: 1) which encodes a protein of 257 amino acid residues (SEQ.ID.NO: 2). The calculated molecular mass of the protein was 30373 Da with 5.35 pl. The greatest similarity of the protein was to Mus musculus dCK (80% identities (213/264), 89% positives (237/264), and 4% gaps (11/264) and Rattus norvegicus dCK (79% identities (210/263), 88% positives (232/263) and 4% gaps (11/263)). This gene was annotated as PZG372 and was named GgdCK1 (Gallus gallus deoxycytidine kinase 1).

The clone pgp2n.pk006.e18 revealed an ORF of 798 bp (SEQ.ID.NO: 3), which encodes a protein of 265 amino acid residues (SEQ.ID.NO: 4). The calculated molecular mass of this protein was 31240 Da with 5.67 pl. The greatest similarity of the protein was to Mus musculus dCK (60% identities (161/265), 81% positives (215/265), and 2% gaps (6/265). Rattus norvegicus dCK showed 60% identities (160/265), 80% positives (213/265) and 2% gaps (6/265). This gene was annotated as PZG378 and was named GgdCK2 (Gallus gallus deoxycytidine kinase 2).

The genes showed 63% identity and 81% similarity between each other, with remarkably different N and C termini (FIG. 2 and FIG. 4). Another variable region is so called insert region. Human dCK contains 15 residues long insert (Ser63-Asn77) which is also found in human dGK but not in TKs or insect dNK enzymes. Similar insert region is present in both chicken genes. However, in GgdCK1 the insert is 16 aa long (Gln56-Ser71) thanks to a unique insertion of an asparagine (Asn57) residue. All amino acids from human dCK involved in the binding of dCyd (Sabini, E et al.: Structure of human dCK suggests strategies to improve anticancer and antiviral therapy. Nat.Struct.Biol. 10:513-519, 2003) are conserved in both chicken genes (FIG. 2).

### Example 2

### Construction of Bacterial Expression Plasmids

This example describes the preparation of bacterial expression plasmids for full-length deoxyribonucleoside kinases. The chicken deoxycytidine kinases were amplified and subcloned as follows:

The ORF of GgdCK1 (SEQ ID NO 1) was amplified by PCR using the primers

ChickendCK1-B:

5′ttaggatccATGGCGACTCCCCCCMGCGCGGGCGGCTGG 3′ (SEQ ID NO: 5), and

ChickendCK1-E:

5′ccggaattcTTATAATGTGCTCAMAATTCCTTCACC 3′ (SEQ ID NO: 6), and using clone pgp1n.pk001.f17 as the template.

The PCR fragment was subsequently cut by EcoRI/BamHI and ligated into pGEX-2T vector (Amersham-Pharmacia) that was also cut by EcoRI/BamHI. The resulting plasmid was named PZG469.

Similarly, the ORF of GgdCK2 (SEQ ID NO 3) was amplified by PCR using the primers

ChickendCK2-B:

5′ ttaggatccATGTCCGCTCCCGCCMGAGGCGCTGCC 3′ (SEQ ID NO: 7), and

ChickendCK2-E:

5′ ccggaattcTTMGMGTCAGGAAAGATTTGATCTCATC 3′ (SEQ ID NO: 8), and using clone pgp2n.pk006.e18 as the template.

In analogy, the PCR fragment was cut by EcoRI/BamHI and subsequently ligated into pGEX-2T vector (Amersham-Pharmacia). The resulting plasmid was named PZG507. This plasmid turned out to have the insert in opposite orientation. A GgdCK2 plasmid with the gene in correct orientation was subsequently made and named PZG657.

For comparison, expression plasmids containing human dCK and Herpes simplex virus TK were also constructed. The deoxycytidine kinase from human was amplified using the primers,

Hs-dCKB:

5′ CGC CGC GGA TCC ATG GCC ACC CCG CCC MG AGA AGC TG 3′ (SEQ ID NO: 9), and, Hs-dCKE:

5′0 CCG GAA TTC TTA CAA AGT ACT CM MA CTC TTT G 3′ (SEQ ID NO: 10), from template plasmid pET-9d dCK provided by prof. Staffan Eriksson.

The PCR fragment was subsequently cut by EcoRI/BamHI and ligated into pGEX-2T vector that was also cut by EcoRI/BamHI. The resulting plasmid was named PZG303

The deoxyguanosine kinase from human without the mitochondrial import sequence was amplified using the forward primer, HudGK-BamHI 5-CGCGGATCCATGGCCAAGAGCCCACTCGAGGGCG-3 (SEQ ID NO. 29) and the reverse primer, HudGK-EcoRI 5-CCGGMTTCTTACAGATTCTTTACAAAGGTGTTTACC-3 (SEQ ID NO. 30) from template plasmid provided by Professor Staffan Eriksson and described in: Eriksson et al, FEBS Lett. Jul. 15, 1996;390(1):39-43. “Cloning and expression of human mitochondrial deoxyguanosine kinase cDNA”.

The PCR fragment was subsequenctly cut by EcoRI/BamHI and ligated into pGEX-2T vector that was also cut by EcoRI/BamHl. The resulting plasmid was named PZG307.

HSV-TK was amplified using the primers, HSV-for A:

5′ CGC GGA TCC ATG GCT TCG TAC CCC GGC CAT C 3′ (SEQ ID NO: 11), and HSV-rev: 5′ CCG GM TTC TTA GTT AGC CTC CCC CAT CTC CCG 3′ (SEQ ID NO: 12), using the plasmid described by Karreman [Christiaan Karreman; Gene 1998 218 57-62] as template. The PCR fragment was cut by EcoRI/BamHI and ligated into EcoRI/BamHl cut pGEX-2T vector. The resulting plasmid was named PZG36.

The ligation mixture was transformed into E. coli strain SURE (Stratagene Cloning Systems, La Jolla, Calif.) the transformed culture was plated on ampicillin media plates and resistant colonies were selected. Plasmid DNA was isolated from transformants, and examined by restriction analysis and sequencing for the presence of the correct fragment (J. Sambrook,. E. Fritsch, T. Maniatis, Molecular Cloning: A Laboratory Manual, Cold Spring Laboratory Press, (1989)).

### Example 3

### Enzyme Activity in Crude Extracts of Chicken Cells, Recombinant Expression and Enzyme Assay

In this example the chicken deoxycytidine kinase enzymes of the invention are expressed and their activity characterised.

Deoxribonucleoside kinase activities in DT 40 chicken cell line DT 40 cells were grown in RPMI-1640 medium (Gibco) supplemented with 10% foetal calf serum, 1% chicken serum, 2 mM L-glutamine, 10 uM mercaptoethanol and penicillin-streptomycin mixture (100U/l), harvested and stored at −80° C. until activity testing. Cells were submitted to brief sonication in extraction buffer (50 mM Tris/HCl pH 7.5, 1 mM DTT, 10% (v/v) glycerol, 1% (v/v) Triton X-100, protease inhibitor cocktail (Complete™ from Roche Diagnostics). Deoxyribonucleoside kinase activities were determined in the DT 40 extracts by initial velocity measurements based on four time samples by the DE-81 filter paper assay using tritium-labelled nucleoside substrates. App. 20 μg extracts were used in the assays. The assay was done as described by Munch-Petersen et al. [Munch-Petersen, B., Knecht, W., Lenz, C., Sondergaard, L. & Piskur, J: Functional expression of a multisubstrate deoxyribonucleoside kinase from Drosophila melanogaster and its C-terminal deletion mutants; J.Biol.Chem. 2000 275 6673-6679].

TABLE 1
Deoxyribonucleoside kinase activity in crude extracts of
DT 40 and MCF7 cells. All assays were performed in triplicates
and the results presented are the mean values with standard
deviation. The four natural deoxyribonucleosides were tested
at a fixed concentration of 200 μM.

| | mU/mg | | | |
|---|---|---|---|---|
| | dThd | dAdo | dGuo | dCyd |
| DT 40 cells | 0.1 ± 0.03 | 0.1 ± 0.01 | 0.09 ± 0.00 | 0.09 ± 0.01* |
| MCF-7 cells | 0.07 ± 0.00 | 0.05 ± 0.00 | 0.01 ± 0.00 | 0.01 ± 0.00 |

*directly measured crude extracts, activity of 0.04 mU/mg was measured for dCyt using crude extract after refreezing.

As can be seen from table 1, crude extracts of chicken cells contained kinases that can phosphorylate all four natural substrates at approximately the same rate as or higher than the corresponding human kinases.

The E. coli strain KY895 (F-, tdk-1, ilv) [Knecht W, Munch-Petersen B and Pi\check{s}kur J: Identification of residues involved in the specificity and regulation of the highly efficient multisubstrate deoxyribonucleoside kinase from Drosophila melanogaster; J. Mol. Biol. 2000 301 827-837] was transformed by various expression plasmids using standard techniques. Transformed KY895 strains were grown to an OD_{600 nm} of 0.5-0.6 in LB/Ampicillin (100 μg/ml) medium at 37° C., and protein expression was induced by addition of 100 μM IPTG. The cells were further grown for 4 hours at 25° C. and subsequently harvested by centrifugation.

Pellets were stored at −80° C. until activity testing. Pellets were submitted to brief sonification in extraction buffer (50 mM Tris/HCl pH 7.5, 1 mM DTT, 10% (v/v) glycerol, 1% (v/v) Triton X-100, protease inhibitor cocktail (Complete™ from Roche Diagnostics).

Deoxyribonucleoside kinase activities were determined in the KY895 extracts by initial velocity measurements based on four time samples by the DE-81 filter paper assay using tritium-labelled nucleoside substrates. 4 to 20 μg extracts were used in the assays. The assay was done as described by Munch-Petersen et al. [Munch-Petersen, B., Knecht, W., Lenz, C., Sondergaard, L. & Piskur, J: Functional expression of a multisubstrate deoxyribonucleoside kinase from Drosophila melanogaster and its C-terminal deletion mutants; J.Biol.Chem. 2000 275 6673-6679].

The protein concentration was determined according to Bradford with BSA as standard protein [Bradford M M: A rapid and sensitive method for the quantitation of microgram quantities of protein utilizing the principle of protein-dye binding; Anal. Biochem. 1976 72 248-254]. SDS-PAGE was done according to the procedure of Laemmli [Laemmi U K: Cleavage of structural proteins during the assembly of the head of bacteriophage T4; Nature 1970 227 680-685], and proteins were visualized by Coomassie staining to verify recombinant protein expression.

The four natural deoxyribonucleosides were tested at a fixed concentration of 200 μM. The results of these experiments are presented in Table 2a below.

Experiments were also performed with another strain of E. coli, TOP10 (Invitrogen). All experimental conditions were the same as for the KY895 cells, except that cells were grown for 5-6 hours after exposure to IPTG. Results are shown in Table 2b.

TABLE 2a
Deoxyribonucleoside Kinase Activity
in Extracts of Transformed KY895.

| Transformant* | dThd | dAdo | dGuo | dCyd |
|---|---|---|---|---|
| pGEX-2T | n.d. | n.d. | n.d. | n.d. |
| PZG469 (pGEX-2T-Gg-dCK1) | 0.55 | 58.83 | 17.94 | 8.02 |
| PZG507 (pGEX-2T-Gg-dCK2) | 0.99 | 0.06 | n.d. | (35.95)** |
| PZG303 (pGEX-2T-Hs-dCK) | | | | |
| PZG449 (pGEX-2T-Hs-dCK mut3) | | | | |
| PZG36 (pGEX-2T-HSV-TK) | 11.84 | 0.23 | 0.54 | 9.98 |

TABLE 2b
Deoxyribonucleoside Kinase Activity in
Extracts of Transformed TOP10 cells.

| Transformant* | dThd | dAdo | dGuo | dCyd |
|---|---|---|---|---|
| pGEX-2T | n.d. | n.d. | n.d. | n.d. |
| PZG469 (pGEX-2T-Gg-dCK1) | 0.30 | 104.11 | 53.67 | 7.83 |
| PZG657 (pGEX-2T-Gg-dCK2) | 0.25 | 290.78 | 213.06 | 15.98 |
| PZG303 (pGEX-2T-Hs-dCK) | n.d. | 6.27 | 6.36 | 1.16 |
| PZG449 (pGEX-2T-Hs-dCK mut3) | 7.30 | 0.22 | 0.24 | 6.15 |
| PZG36 (pGEX-2T-HSV-TK) | 11.84 | 0.23 | 0.54 | 9.98 |

*pGEX-2T is the vector and is available from Amersham-Pharmacia;
*PZG469 (pGEX-2T-Gg-dCK1) is the vector containing the gene encoding a chicken dCK1 enzyme;
*PZG507 (pGEX-2T-Gg-dCK2) is the vector containing a gene encoding a chicken dCK2 enzyme in opposite orientation;
*PZG657 (pGEX-2T-Gg-dCK2) is the vector containing a gene encoding a chicken dCK2 enzyme;
*PZG303 (pGEX-2T-Hs-dCK) is the vector containing a gene encoding a human dCK enzyme;
*PZG449 (pGEX-2T-Hs-dCK mut3) is the vector containing a gene encoding a human dCK enzyme containing 3 mutations (Sabini E, Ort S, Monnerjahn C, Konrad M, Lavie A. Nat Struct Biol. 2003 10: 513-9.)
*PZG36 (pGEX-2T-HSV-TK) is the vector containing a gene encoding a Herpes virus TK enzyme;
**High activity of PZG507 for dCyd was due to experimental artefact;
The numbers show the specific activity in mU/mg (pmol/mg of protein/min).
n.d. = not detectable (activity below detection limit of 0.5 pmol/min/mg).

In KY895 cells, the dCK1 from Gallus gallus (PZG469) was able to phosphorylate dCyd, dAdo and dGuo, but not dThd. Surprisingly dAdo was the best substrate followed with dGuo and dCyd. In contrast, dCK2 (PZG507) phosphorylated dCyd with the highest efficiency but this particular result could not be reproduced.

In TOP10 cells, human dCK phosphorylated dGuo and dAdo equally well, while dCyd phosphorylation was much lower.

In TOP10 cells, the dCK1 from Gallus gallus (PZG469) was able to phosphorylate dCyd, dAdo and dGuo, but not dThd. Surprisingly dAdo was the best substrate followed with dGuo and dCyd. dCK2 (PZG657) also phosphorylated dAdo with the highest efficiency. In contrast to GgdCK1, GgdCK2 phosphorylated dCyd with double efficiency compared to GgdCK1 (app. 16 mU v.s. app. 8 mU).

As the encoded enzymes were comparable to human dCK in their activity, this further supported the classification of both enzymes as deoxycytidine kinases.

### Example 4

### Determination of LD₁₀₀ of Transformed KY895

Deoxyribonucleoside kinases are of interest as suicide-genes to be used in gene-mediated therapy of cancer or viral infections. In this example, the potential of the chicken dCK kinases of the invention to convert different nucleoside analogs are compared to that of the human Herpes simplex virus type 1 thymidine kinase (HSV1-TK) and the human deoxycytidine kinase (Hs-dCK) in a bacterial test system.

The experiment was carried out essentially as described by Knecht et al. [Knecht W, Munch-Petersen B and Piskur J: Identification of residues involved in the specificity and regulation of the highly efficient multisubstrate deoxyribonucleoside kinase from Drosophila melanogaster; J. Mol. Biol. 1970 301 827-837]. Briefly, overnight cultures of transformed KY895 were diluted 200-fold in 10% glyercol and 2 μl drops of the dilutions were spotted on M9 minimal medium plates [Ausubel F, Brent R, Kingston R E, Moore D D, Seidman J G, Smith J A & Struhl K (Eds.): Short protocols in molecular biology; 3^{rd} edition (1995) pp.1-2, Wiley, USA] supplemented with 0.2% glucose, 40 μg/ml isoleucine, 40 μg/ml valine, 100 μg/ml ampicillin and with or without nucleoside analogs. Growth was inspected visually after 24 hours of incubation at 37° C.

The results of the experiment are presented in Table 3a below.

Experiments were also performed with another strain of E. coli, TOP10 (Invitrogen). All experimental conditions were the same as for the KY895 cells, except that cells were grown for 5-6 hours after exposure to IPTG. Results are shown in Table 3b.

TABLE 3a
LD₁₀₀ Values for Growth of Transformed KY895 Cells on
Nucleoside Analog dFdC and Deoxyribonucleoside Kinase
Specific Activity in KY895 Extracts towards dFdC.

| | LD₁₀₀ | mU/mg |
|---|---|---|
| Transformant* | dFdC (μM) | dFdC |
| pGEX-2T | 100 | n.d. |
| PZG469 (pGEX-2T-Gg-dCK1) | 0.1 | 16.44 |
| PZG507 (pGEX-2T-Gg-dCK2) | | |
| PZG303 (pGEX-2T-Hs-dCK) | 100 | |
| PZG449 (pGEX-2T-Hs-dCK mut3) | 100 | |
| PZG36 (pGEX-2T-HSV-TK) | 100 | 1.85 |

*See comments to Table 2.

TABLE 3b
LD₁₀₀ Values for Growth of Transformed TOP10 Cells on
Nucleoside Analog dFdC and Deoxyribonucleoside Kinase
Specific Activity in TOP10 Extracts towards dFdC.

| | LD₁₀₀ | mU/mg |
|---|---|---|
| Transformant* | dFdC (μM) | dFdC |
| pGEX-2T | 100 | n.d. |
| PZG469 (pGEX-2T-Gg-dCK1) | 0.1 | 20.26 |
| PZG657 (pGEX-2T-Gg-dCK2) | 0.3 | 11.84 |
| PZG303 (pGEX-2T-Hs-dCK) | 0.1 | 9.31 |
| PZG449 (pGEX-2T-Hs-dCK mut3) | 0.05 | 7.17 |
| PZG36 (pGEX-2T-HSV-TK) | 100 | 1.85 |

*See comments to Table 2.

As can be seen from Table 3a, PZG469 was the most efficient chicken kinase, as reflected by the lowest LD₁₀₀, in killing KY895 on dFdC plates and the highest specific activity for gemcitabine. The LD₁₀₀ was 1000-fold lower than that of human dCK, which sensitised the cells to the same degree as the empty plasmid pGEX-2T.

In TOP10 cells (Table 3b), PZG469 was the most efficient chicken kinase, as reflected by the lowest LD₁₀₀, in killing TOP10 cells on dFdC plates and the highest specific activity for gemcitabine. The LD₁₀₀ was 1000-fold lower than that of the empty plasmid pGEX-2T.

TOP10 cells transfected with the expression plasmid for PZG657 could be killed at 300-fold lower concentrations than cells transformed with pGEX-2T-HSV-TK or pGEX-2T.

Using similar methods, KY895 cells transformed with different deoxycytidine kinases were plated on medium containing increasing concentrations of the nucleoside analogue Ara-G. Apart from the two chicken kinases, human dCK and human dGK was used as controls. The results are shown in table 3C.

TABLE 3C
Killing of Transformed KY895 Cells growing in the presence of Nucleoside Analog ara-G.

| KY895 TK- | LB | 100 nM | 316 nM | 1 μM | 3.16 μM | 10 μM | 31.6 μM | 100 μM |
|---|---|---|---|---|---|---|---|---|
| pGEX-2T | +++ | +++ | +++ | +++ | +++ | +++ | +++ | +++ |
| pZG307 Hs dGK | +++ | +++ | +++ | +++ | +++ | +++ | +++ | +++ |
| pZG333 Hs dCK | +++ | +++ | +++ | +++ | +++ | +++ | +++ | +++ |
| pZG469 Gg dCK1 | +++ | +++ | +++ | +++ | +++ | +++ | +++ | +++ |
| PZG657 Gg dCK2 | +++ | +++ | +++ | +++ | +++ | −−− | −−− | −−− |

As can be seen from Table 3C, Chicken dCK2 (dAK) gene was the most efficient in phosphorylating ara-G, as reflected by the lowest LD₁₀₀, and thereby in killing KY895 on ara-G plates. The LD₁₀₀ was at least 10-fold lower than that of chicken dCK1, human dGK and human dCK, which sensitised the cells to the same degree as the empty plasmid pGEX-2T. Due to the low solubility of ara-G it was not possible to determine LD₁₀₀ for KY895 cells transformed with the empty vector, Hs dCK, Hs dGK, and GgdCK1.

### Example 5

### Construction of a Retrovirus Vector Expressing Chicken Deoxycytidine Kinases

The cDNA of chicken dCK kinases were cloned into a retrovirus vector based on the Moloney murine leukemia (MLV) virus to generate a replication-deficient recombinant retrovirus containing the kinases.

All DNA fragments were amplified with Pfu polymerase (Stratagene) using primers with designed flanking restriction enzyme sites and containing Kozak sequence at 5′ end.

Constructs were cut either with Xhol I BglII (chicken dCK1 and human dCKs) or Sall I BamHI (chiken dCK2) and cloned into the XhoI-BglII site of the pLCXSN plasmid vector (NsGene A/S) under the control of CMV promoter.

The constructs obtained were named as: PZG460 (chicken dCK1-PLCXSN), PZG529 (chicken dCK2-PLCXSN), PZG309 (human dCK-PLCXSN) and PZG463 (human dCK-mut3-PLCXSN). pLCXSN alone was used as a control.

The plasmids were purified using the Qiagen plasmid kit (QIAGEN) and DNA sequences of the constructed plasmids were verified by DNA sequence determination.

The following primer sequences were used:

| | |
|---|---|
| Chiken dCK1 + Kozak XhoI vir | |
| 5-tccctcgaggccaccatggcgactcccccca | (SEQ ID NO. 13) |
| agcgcgg-3 | |
| Chiken dCK1 + BglII vir | |
| 5-GAAGATCTTCATAATGTGCTCAAAAATTCCT | (SEQ ID NO. 14) |
| TCAC-3 | |
| Chiken dCK2 + Kozak SalI vir | |
| 5′-ACGCGTCGACGCCACCATGTCCGCTCCCGC | (SEQ ID NO. 15) |
| CAAGAGG-3′ | |
| Chiken dCK2 + BamHI vir | |
| 5′-CGGGGATCCTCAAGAAGTCAGGAAAGATTT | (SEQ ID NO. 16) |
| GATCTC-3′ | |
| Human dCK + Kozak XhoI vir | |
| 5′-CCGCTCGAGGCCACCatggccaccccgccc | (SEQ ID NO. 17) |
| aagagaagctg-3′ | |
| Human dCK + BglII vir | |
| 5′-gaagatcttcacaaagtactcaaaaactct | (SEQ ID NO. 18) |
| ttg-3′ | |
| Human dCK mut3 + Kozak XhoI vir | |
| 5′-CCGCTCGAGGCCACCatggccaccccgccc | (SEQ ID NO. 19) |
| aagagaagctg-3′ | |
| Human dCK mut3 + BglII vir | |
| 5′-gaagatcttcacaaagtactcaaaaactct | (SEQ ID NO. 20) |
| ttg-3′ | |

HE 293 T packaging cells (ATCC CRL-11268) were cultured at 37° C. in OPTIMEM 1 medium (Life Technologies, Inc.) The constructed pLCXSN plasmid vector was transfected into the packaging cells using LipofectAMINE PLUS (Life Technologies, Inc.) according to the protocol provided by the supplier. The medium from the transfected cells was collected 48 hours after transfection, filtered through a 0.45 pm filter, pelleted by ultracentrifugation (50.000×g, 90 minutes at 4° C.) and dissolved in DMEM (Cambrex, Blo Whittaker Cat. No. 12-741-F).

The virus containing medium was subsequently used to transduce the cancer cell lines with a MOI of 5.

Cell Culture and Retroviral Transduction

Human breast MCF-7 (ATCC HTB-22) and Glioblastoma U-1 18-MG (ATCC HTB-15) cancer cells were purchased from the American Type Culture Collection. Cells were cultured in RPMI, E-MEM or D-MEM (Cambrex, Bio Whittaker Cat. No. 12-115-F, 12-611 and 12-741-F) with 10% (v/v) Australian originated fetal calf serum (Cambrex, Bio Whittaker Cat. No. 12-611) and 1 ml/l of Gentamicin (Cambrex, Bio Whittaker Cat. No. 17-518). Cells were grown at 37° C. in a humidified incubator with a gas phase of 5% CO₂.

The cells were transduced with the retrovirus containing medium mixed with 5 μg/ml of Polybrene, incubated for 48 hours and then cultured continuously for 3 weeks in the presence of 300-400 μg/ml Genetecin® (Life Technologies Inc.).

Cell Proliferation Assay—Cytotoxicity

Cells were plated at densities range of 1.500-3.500 cells/well in 96-well plates coated with Poly-L-lysine (Sigma Cat. No. P6282) Gemcitabine (obtained from Orifarm A/S—DK) was added after 24 hours of incubation at 37° C., 5% CO₂, and the medium containing the nucleoside analog. Each experiment was performed in four replicates. Cell survival was assayed after 96-120 hours of drug exposure, by XTT cell proliferation kit (XTT kit II, Roche Cat. No. 1 465 015). The data was corrected for background media-only absorbance where after the 50% cell killing drug concentration—(IC₅₀ value) was calculated. The IC₅₀ value of the investigated drug/compound was calculated as the mean of these experiments using SigmaPlot® (SPSS Science, Dyrberg Trading—DK).

Expression of Chicken dCKs in Human Cells

The sensitivity of the untransduced cells, and of the cells transduced with either the retroviral vector alone ore the vector containing chicken kinases for Gemcitabine and Ara-G was determined.

The cytotoxicity (IC₅₀ value) was determined after 96-120 hours of drug exposure. The results are presented in the table below.

TABLE 4a
Sensitivity (IC₅₀) of the MCF-7 (breast cancer cell line)
to Gemcitabine. The concentrations which cause 50% lethality
are shown for each construct and the parental cell line.
The factor of sensitivity increase is compared to the cell
line containing the empty vector pLCXSN.

| MCF-7 | IC₅₀ value (mM) | Sensitivity factor |
|---|---|---|
| pLCXSN | 2.2136 ± 0.2891 | — |
| PZG 460 (Chicken dCK1) | 0.00043 ± 0.00012 | 5148-fold |
| PZG 529 (Chicken dCK2) | 0.0696 ± 0.0186 | 32-fold |
| PZG 309 (Human dCK) | 0.0522 ± 0.0204 | 42-fold |
| PZG 463 (Human dCK mut3) | — | — |

The difference in sensitivity between the parental cell line and the cells transduced with the pLCXSN vector alone was less than 1-fold. The breast cancer cell line, that expressed the different chicken kinases, showed an increase in sensitivity to Gemcitabine. The highest increase was detected in for the cells expressing the Chicken dCK1 kinase with a more than 5.000-fold decrease in IC₅₀ value compared to the cells expressing the empty vector (pLCXSN). Chicken dCK2 kinase led to a 32 fold decrease in IC₅₀ value for gemcitabine. At the same time human dCK lead to 42-fold sensitivity increase, while the improved human dCK mutant did not show any sensitivity improvements.

Arabinofuranosylguanine (Ara-G) is an analog of deoxyguanosine, which was synthesized as early as in 1964. The clinical use of Ara-G was hampered for many years by its low solubility until a soluble prodrug of Ara-G (nelarabine) was synthesized. It is an arabinosyl nucleoside, resistant to purine nucleoside phosphorylase (PNP) with proven activity against various refractory hematological malignancies such as T-cell acute lymphoblastic leukemia, T-lymphoid blast crisis, T-lymphoma, and B-cell chronic lymphocytic leukemia.

Deoxyguanosine kinase (dGK) is a nucleoside kinase located in the mitochondria. It catalyzes the phosphorylation of deoxyguanosine and deoxyinosine but is also known to carry out the first, rate-limiting, step in the activation of several nucleoside analogs. Among these, Ara-G has shown the highest affinity for human dGK with a Km close to that of deoxyguanosine (8.0 μM compared to 7.6 μM) and relative phosphorylation of Ara-G showed to be 76 times higher by recombinant human dGK than by recombinant human dCK. Ara-G is further activated to its cytotoxic metabolite, Ara-G triphosphate (Ara-GTP), which inhibits DNA polymerase and ribonucleotide reductase and is incorporated into DNA, terminating DNA chain elongation, resulting in cell death. The metabolism and mechanism of the action of Ara-G have shown similarities to those of other deoxynucleosides (Zhu. et al, J.Biol.Chem., 273:14707-14711 (1998)).

Chicken dCK2 showed to be very good activator ara-G in the human breast cancer cell line MCF-7. Compared to parental cell line, retrovirus transduced MCF-7 expressing chicken dCK2 gene showed app. 350 fold increase in sensitivity towards ara-G (see Table 4b).

TABLE 4b
Sensitivity (IC₅₀) of the MCF-7 (breast cancer cell line) to
ara-G. The concentrations which cause 50% lethality are shown
for each construct and the parental cell line. The factor of
sensitivity increase is compared to the parental cell line.

| MCF-7 | IC₅₀ value (mM) | Sensitivity factor |
|---|---|---|
| parental cell line | 1.582 ± 0.1583 | — |
| PZG 529 (Chicken dCK2) | 0.00477 ± 0.00138 | 332-fold |

### Example 6

### Kinetic Data for Chicken dCKs Enzymes

Purification of the Recombinant Enzymes. Transformants (KY895 cells transfected with PZG469 (GgcCK1) and PZG657 (GgdCK2)) were grown in LB medium containing 100 mg/ml ampicillin to A₆₀₀ 0.5-0.6, and the expression was induced with 100 mM IPTG for 4 h at 25° C. The cells were harvested by centrifugation, and the pellet was resuspended in 25 ml ice-cold binding buffer A (20 mM NaPO₄ pH 7,3; 150 mM NaCl; 10 % Glycerol; and 0,1% Triton X-100) containing protease inhibitor cocktail (Complete™—EDTA free from Roche Diagnostics). The cells were homogenized using a French Press, subjected to centrifugation at 12,000×g for 30 minutes (4° C.), filtered through a 1 mm Whatman glass microfiber filter and a 0.45 mm cellulose acetate filter, and loaded onto the column (2 ml column packed with Glutathione-Sepharose 4 FF, Pharmacia) equilibrated in binding buffer A. Unbound material was removed by washing with 10 column volumes of buffer A. Subsequently the column was washed with 5 ml buffer A containing 10 mM ATP/MgCl₂, and incubated for 1 h at room temperature and then 30 min at 4° C. to remove strongly bound contaminating proteins. Afterwards the column was washed again with 10 ml of buffer A and 2 ml of thrombin (50 U/ml) solution were applied on the column. The column was gently shaken overnight at 4° C. to cleave the recombinant protein from the GST-tag. Pure protein was eluted from the column in buffer A. Uncleaved fusion protein was eluted with buffer B (50 mM Tris-HCl pH 8, 10% glycerol, 0,1 % Triton X-100, 10 mM gluthatione reduced). Before storage of enzyme-containing fractions at −80° C., glycerol, Triton X-100, and dithiothreitol were added to 8%, 1% and 1 mM, respectively. Proteins were analyzed on Agilent 2100 Bioanalyzer using Protein Assay Chips.

Enzyme Assays. Bacteria or cell lines were grown as described, harvested and stored at −80° C. until activity testing. Cells were submitted to brief sonication in extraction buffer (50 mM Tris/HCl pH 7.5, 1 mM DTT, 10% (v/v) glycerol, 1% (v/v) Triton X-100, protease inhibitor cocktail Complete™ from Roche Diagnostics). Deoxyribonucleoside kinase activities were determined by initial velocity measurements based on four time samples by the DE-81 filter paper assay using tritium-labelled nucleoside substrates. App. 20 μg extracts were used in the assays. The assay was done as described in Munch-Petersen et al. [Munch-Petersen, B., Knecht, W, Lenz, C., Sondergaard, L. & Piskur, J: Functional expression of a multisubstrate deoxyribonucleoside kinase from Drosophila melanogaster and its C-terminal deletion mutants; J.Biol.Chem. 2000 275 6673-6679]. One unit of deoxyribonucleoside kinase activity is defined as 1 μmol of the corresponding monophosphate formed per minute. The obtained data were fitted to the Michaelis-Menten equation v=Vₘₐₓ·[S]/(Kₘ+[S]).

Both proteins were purified to homogeneity using GST tagged protein constructs. According to measurements obtained with Agilent 2100 Bioanalyzer GgdCK1 was purified to 99% with calculated size of 28.6 kDa. GgdCK2 protein was 74% pure and preparation contained some uncut GST-dCK2 protein. A size of 28.1 kDa was determined for GgdCK2.

Human dCK phosphorylates dCyd most efficiently, but also dAdo and dGuo, using UTP, ATP or other nucleoside triphosphates as phosphate donors. The relation between velocity and substrate concentration was determined for several deoxyribonucleosides and their analogs (Table 5). The GgdCK1 uses dCyd as the best substrate with a Km of 4.8 μM. Although dAdo gave the highest turnover rate (Kcat=0.48/s), the specificity constant Kcat/Km showed that dCyd was the most efficient substrate, almost 20-fold better than dGuo. The dCyd analog gemcitabine was catalyzed with the highest specificity (2,2×10⁵ M/s), followed with dAdo analog cladribine (1,9×10⁵ M/s).

GgdCK2 also phosphorylated dCyd with the lowest Km but with very low turnover rate (Kcat=0.03/s) and low efficiency. However, dAdo was the most efficient substrate with highest turnover rate (Kcat/s) and specificity constant (Kcat/Km). This was even more evident in one replicate, where the specificity constant for dAdo was 1.0×10⁶. In addition this enzyme phosphorylated dAdo analogs fludarabine (F-araA) and cladribine (CdA) with much higher efficiency than dCyd analogs such as gemcitabine and araC (see Table 5). Considering the presented kinetic properties of the GgdCK2, which showed highest catalytic efficiency for dAdo and dAdo-based analogs, we propose that this enzyme may represent a deoxyadenosine kinase (dAK) in correlation with the nomenclature for the other broad substrates deoxynucleoside kinases such as dCK. The chicken dCK2 enzyme has broad substrate specificity similar to dCK and belongs structurally and phylogenetically to the dCK/dGK/TK2 family of deoxynucleoside kinases. The GgdCK2 therefore represents the first example of a dAK from any eukaryotic species.

TABLE 5
Steady state kinetic data for chicken dCK1 and dCK2 enzymes.

| | | Vmax | | |
|---|---|---|---|---|
| Nucleoside | Km (μM) | nm/min/mg | Kcat/s | Kcat/Km (M s) |
| GgdCK1 | | | | |
| dCyd | 4.8 ± 0.9 | 589 ± 34 | 0.30 | 6.3 × 10⁴ |
| dAdo | 79.0 ± 18.0 | 960 ± 124 | 0.48 | 6.1 × 10³ |
| | 82 ± 8 | 3839 ± 123 | 2.10 | 2.6 × 10⁴ |
| dGuo | 61.8 ± 11.1 | 394 ± 37 | 0.20 | 3.2 × 10³ |
| Gemcitabine | 6.3 ± 0.5 | 2712 ± 357 | 1.37 | 2.2 × 10⁵ |
| F-araA | 277 ± 33 | 6649 ± 295 | 3.36 | 1.2 × 10⁴ |
| araC | 15.3 ± 2.3 | 878 ± 33 | 0.44 | 2.9 × 10⁴ |
| CdA | 3.3 ± 0.4 | 1273 ± 38 | 0.64 | 1.9 × 10⁵ |
| GgdCK2 | | | | |
| dCyd | 3.4 ± 1.2 | 51 ± 5 | 0.03 | 8.8 × 10³ |
| dAdo | 60 ± 4.6 | 11887 ± 400 | 6.18 | 1.0 × 10⁶ |
| | 141 ± 16 | 6995 ± 236 | 3.74 | 2.7 × 10⁴ |
| dGuo | 17 ± 4 | 1481 ± 96 | 0.77 | 4.5 × 10⁴ |
| Gemcitabine | 311 ± 61 | 988 ± 137 | 0.51 | 1.6 × 10³ |
| F-araA | 180 ± 29 | 4547 ± 248 | 2.36 | 1.3 × 10⁴ |
| araC | 1469 ± 664 | 649 ± 267 | 0.34 | 2.3 × 10² |
| CdA | 3.8 ± 0.6 | 1281 ± 50 | 0.67 | 1.8 × 10⁵ |
| HsdCK* | | | | |
| dCyd | 1 | 200 | 0.11 | 1.1 × 10⁵ |
| dAdo | 80 | 800 | 0.44 | 5.5 × 10³ |
| dGuo | 100 | 600 | 0.33 | 3.3 × 10³ |
| Gemcitabine | 60 | 100 | 0.05 | 1.0 × 10³ |
| F-araA* | 200 | 100 | 0.05 | 1.0 × 10³ |
| araC | 20 | 400 | 0.22 | 1.1 × 10⁴ |
| CdA | 2 | 200 | 0.11 | 5.5 × 10⁴ |

*Data from Bohman, C. and Eriksson, S.: Deoxycytidine kinase from human leukemic spleen: preparation and characteristics of homogeneous enzyme. Biochemistry 27: 4258-4265, 1988. and Habteyesus, A., Nordenskjold, A., Bohman, C., and Eriksson, S.: Deoxynucleoside phosphorylating enzymes in monkey and human tissues show great similarities, while mouse deoxycytidine kinase has a different substrate specificity. Biochem. Pharmacol. 42: 1829-1836, 1991.

### Example 7

### Random Mutagenesis and Screening of Improved Mutant of dCK1

Random PCR mutagenesis. The random mutagenesis PCR procedure was performed as described in Zaccolo et aL. [Zaccolo, M., Williams, D. M., Brown, D. M., and Gherardi, E. (1996) J.MoLBioL 255, 589-603] with some modifications. PCR mutagenesis reaction contained template expression-vector PZG469 (10 fmol), and primers:

| | |
|---|---|
| P209 | |
| 5′ TTAGGATCCATGGCGACTCCCCCCAAGCGC | (SEQ ID NO. 21) |
| GGGCGGCTGG 3′ | |
| and | |
| P210 | |
| 5′ CCGGAATTCTTATAATGTGCTCAAAAATTC | (SEQ ID NO. 22) |
| CTTCACC 3′ | |

with 20 pmol each, and dNTPs at 0.2 mM each. The nucleotide analogs dPTP and 8-oxo-dGTP were present at 5 μM. PCR conditions were: denaturation at 95° C. for 5 min., 15 cycles with 95° C. for 45 sec., 50° C. for 45 sec., 72° C for 75 sec. and finally prolongation at 72° C. for 10 min. The PCR products were purified with the PCR purification kit from Boehringer-Mannhein and eluted in 200 μl of 5 mM Tris-HCl (pH 7.5): 30 μl of this eluate was used in the second PCR without nucleotide analogs, which was done in a volume of 100 μl with 0.5 unit of Taq polymerase, 65 pmol of each primer, 0.2 mM each dNTP. PCR conditions were the same as in the first PCR. The mutagenized PCR fragments were again purified, cut with BamHI and EcoRI, and subcloned into the pGEX-2T plasmid vector. The TOP10 E. coli strain (InVitrogen) was electrotransformed with the ligation mix, using standard techniques, and plated on LB-ampicillin (100 mg/ml) plates. The degree of mutagenicity was determined by sequencing of randomly picked clones. Selection of mutants was done on M9 minimal medium plates containing 0, 5, 50 and 100 nM of gemcitabine. Plates were prepared by mixing the medium at 56° C. with the gemcitabine, before pouring the plates. Growth of colonies was visually inspected after 24 hours at 37° C. From clones not growing on nucleoside analog-containing plates, but growing normally on plates without the nucleoside analog, the plasmid was isolated and retransformed into TOP10. These clones were retested to verify the plasmid-borne phenotype. All clones with increased sensitivity towards gemcitabine were tested again on plates with logarithmic dilutions of the nucleoside analog to determine the lethal doses (LD₁₀₀) of the analog, at which no growth of bacteria could be seen. Plates with the concentration ranges 5-100 nM dFdC were used to determine the LD₁₀₀ value of putative mutants. Screening of the mutant library from chicken dCK1. After transformation of ligation mix and incubation over night colonies were picked and inoculated into individual wells of a 384-well plate containing 100 μl LB medium and 100 μg/ml ampicillin per well. Five wild type colonies were included per plate. The addition of 10% glycerol to the media allows direct freezing at −80° C. 384-well plates with the mutant clones were collected in mutant libraries—one library for each mutation condition—and stored at −80° C. The colonies were incubated overnight at 37° C., shaking. Cultures were then replicated on freshly prepared medium, containing different concentrations of gemcitabine, with a 384-pin replicator and were incubated again over night at 37° C. The lethal dose of each individual mutant was determined as the concentration for lack of growth. Mutants, expressing an improved dCK activity, were identified by visual inspection and by comparison to LD₁₀₀ of wild-type colonies. Selected mutant plasmids were transformed by heat shock into E. coli cells. Three colonies from each transformation were incubated separately in 100 μl medium in a 96-well microtiter plate for 2.5 hours. Three microliters of each culture was spotted with a multi-pipette on plates containing 30 ml solid a-minimal medium supplemented with gemcitabine in following concentrations: 2.5, 5, 7.5, 10, 15, 20, 25, 30, 40, 50, 60, 80 and 100 nM. Colonies containing the wild-type chicken dCK gene were added as references. Mutants showing a 2.5-3 fold lower lethal dose than wild-type colonies at a lethal dose of 30 nM and below were plasmid purified, heat shock transformed and upcoming clones were subjected to the final activity test. After screening, clones were stored in glycerol stocks in microtiter plates at −80° C. until needed.

Determination of LD₁₀₀ of chicken dCK wild-type gene. The lethal dose of the colony containing the wild type dCK sequence serves as a reference value to compare it with the mutants obtained in the mutant library. Therefore, multiple tests have to be performed to investigate the lethal dose of dCK wild type colonies. Only clones exhibiting a lethal dose significantly below the lethal dose of wild-type clones were of interest and were taken into consideration as good required mutants. Several clones of wild-type genes had always to be included due to differences in the performance of the experiments. The difference in LD₁₀₀ among the wild-type colonies of dCK1 could be explained by slight experimental variations during the performance. Therefore at least three colonies were analyzed in the final test, which were based not only on the wild-type genes, but also on the selected mutant genes. The lethal dose of wild type colonies was found in the range of 60 to 100 nm gemcitabine, where most lethal concentrations were found at 80 nM.

From each library (A, B, C and D) approximately 4300 clones were screened on gemcitabine to investigate which library showed the most promising results in respect to further screening. Highly active clones, which sensitize transformed competent E. coli host cells to gemcitabine were found in the library created under condition C, based on 2.5 μM nucleotide analogues and 15 PCR cycles. Condition C represented the lowest mutational load of all four conditions. The sequencing results, showing the mutated positions for each clone are presented in Table 6. The lethal dose LD₁₀₀ for every clone was estimated as well, compared to wild type dCK.

The lowest lethal doses of individual mutant clones, selected, were in the range of 10 nM to 30 nM gemcitabine. Mutations occurred at 35 different positions in the peptide sequence. At six positions a mutation was found in more than one clone. Whether these positions likely play an important role for the LD₁₀₀ value observed, is not quite clear. Position El I was targeted seven times, position Y184 three times and the four positions P4, M79, K82 and F129 were mutated twice. In these 16 selected mutants 47 amino acid shifts were observed, which corresponds to an average of almost three amino acid mutations per protein. This value was found to be slightly below the average value based on mutation condition A (25 PCR cycles and 2.5 nM nucleotide analogues) with 3.16 changes per protein and was found above the value of condition C (15 PCR cycles and 2.5 nM nucleotide analogues) with 2.66 amino acid mutations in average.

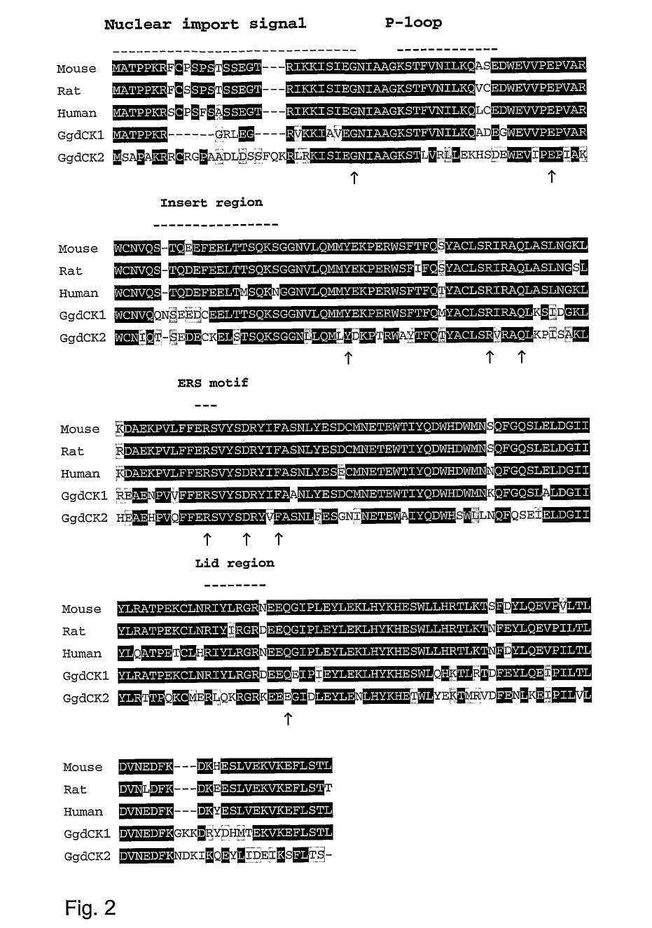

One mutant clone under condition C was found, where two dCK genes behind each other were observed by sequencing. The plasmid, carrying the double gene, was included into the plasmid collection and was named pZG634. The lethal dose LD₁₀₀ of the double gene pZG634 showed a very low value, compared to all other mutants selected, with a factor of 3-4 times lower corresponding to a LD₁₀₀ value of ca. 2.5 nM. The double gene was created by a deletion mutation of the stop codon TAA, which was mutated to GGA (glycine), followed by the nucleotide sequence TCC, which encodes a serine. Right after the amino acid serine the peptide sequence of dCK was starting again with the start codon ATG encoding methionine. The decisive part of the nucleotide and peptide sequence of the double gene is compared to the wild type sequence in FIG. 3. Both sequences in one plasmid could be verified by using forward and reverse pGEX-2T primers for the same plasmid, which had the double gene insert. It is worth mentioning, that the nucleotide changes at the end of the first protein replaced the stop codon and introduced a BamHl restriction site (GGATCC) instead. The double gene was investigated in detail, in order to find out, which gene or which mutation caused the high sensitivity of the E. coli host cell towards gemcitabine. For this purpose, the two linked genes were separated and different clones carrying the genes separately were tested for their LD₁₀₀ value, in order to determine the essential part responsible for this low LD₁₀₀ value. After the directed mutation could be verified by sequencing, the LD₁₀₀ values of colonies carrying both genes individually were investigated. This low LD₁₀₀ in the range of 5 nM, as observed for the double mutant, could neither be observed in clones carrying the first gene, nor in clones carrying the second gene. The clone, containing only gene A expressed a lethal dose of around 30 nM gemcitabine, whereas the clone with only gene B had a LD₁₀₀ value towards gemcitabine in the range of 60 nM similar to wild type dCK1 colonies.

TABLE 6
Mutations in chicken dCK1 that sensitise transformed Top 10 E. coli cells to
the nucleoside analogue gemcitabine and their LD₁₀₀ values. The position
of amino acid exchanges for each mutant is shown including the respective
wild type amino acid. An asterisk (*) indicates sites that are mutagenised
in more than one mutant. For the double gene (pZG634) both sequences are shown,
whereas the first gene (654) has one mutation shift and the second gene (655)
represents four amino acid changes. (A) indicates first gene of double
mutant and (B) indicates second gene of double mutant, respectively. Numbers in
the first column represent the plasmids included in the plasmid collection.

| | amino acid pos. | | | | | | | | | | | | | | | |
|---|---|---|---|---|---|---|---|---|---|---|---|---|---|---|---|---|
| | 4 | 8 | 11 | 12 | 49 | 50 | 54 | 59 | 60 | 68 | 71 | 73 | 74 | 79 | 82 | 90 |
| dCK1 | P | E | E | G | A | R | V | E | E | S | S | G | N | M | K | F |
| PZG634 | | | | | | | | | | | | | | | | |
| PZG654 | | | | | | | | | | | | | | | | Y |
| (A) | | | | | | | | | | | | | | | | |
| PZG655 | | | G | | | | | | | | | | | | E | |
| (B) | | | | | | | | | | | | | | | | |
| PZG635 | | | G | D | V | | | | | | | | | | | |
| PZG636 | | | | | | | | | | | | | S | | | |
| PZG637 | | | | | | | | G | | | | | | T | | |
| PZG638 | | | | | | | | | G | | | R | | | R | |
| PZG639 | | | G | | | | | | | | I | | | | | |
| PZG640 | L | | G | | | | | | | | | | | | | |
| PZG641 | | | G | | | | | | | | | | | T | | |
| PZG642 | | G | | | | | | | | | | | | | | |
| PZG643 | | | | | | | | | | P | | | | | | |
| PZG644 | | | | | | | | | | | | | | | | |
| dCK1 | P | E | E | G | A | R | V | E | E | S | S | G | N | M | K | |
| PZG645 | | | | | | | | | | | | | | | | |
| PZG646 | | | G | | | | | | | | | | | | | |
| PZG647 | | | | | | | A | | | | | | | | | |
| PZG648 | | | G | | | G | | | | | | | | | | |
| PZG649 | L | | | | | | | | | | | | | | | |
| | * | | * | | | | | | | | | | | * | * | |

| | amino acid pos. | | | | | | | | | | |
|---|---|---|---|---|---|---|---|---|---|---|---|
| | 92 | 99 | 103 | 112 | 115 | 139 | 147 | 156 | 158 | 177 | 183 |
| dCK1 | M | I | L | E | N | D | T | M | K | E | I |
| PZG634 | | | | | | | | | | | |
| PZG654 | | | | | | | | | | | |
| (A) | | | | | | | | | | | |
| PZG655 | | V | P | | | | | | | | |
| (B) | | | | | | | | | | | |
| PZG635 | | | | | S | | | | | | |
| PZG636 | | | | | | | | | | | |
| PZG637 | | | | | | | | | | | |
| PZG638 | | | | | | | | | | | |
| PZG639 | V | | | | | | | | | | |
| PZG640 | | | | | | | S | | | | |
| PZG641 | | | | | | G | | | | | |
| PZG642 | | | | | | | | | | | |
| PZG643 | | | | | | | | | E | | |
| PZG644 | | | | | | | | T | | | |
| dCK1 | M | I | L | E | N | D | T | M | K | E | I |
| PZG645 | | | | G | | | | | | | T |
| PZG646 | | | | | | | | | | | |
| PZG647 | | | | | | | | | | K | |
| PZG648 | | | | | | | | | | | |
| PZG649 | | | | | | | | | | | |

| | amino acid pos. | | | | | | | | |
|---|---|---|---|---|---|---|---|---|---|
| | | | | | | | | | LD₁₀₀ |
| | 184 | 189 | 190 | 194 | 204 | 219 | 239 | 247 | (nM) |
| dCK1 | Y | D | E | I | Y | F | K | T | 80 |
| PZG634 | | | | | | | | | 2.5-5 |
| PZG654 | | | | | | | | | 30 |
| (A) | | | | | | | | | |
| PZG655 | | | | | | | | | 60 |
| (B) | | | | | | | | | |
| PZG635 | | | | | | C | | I | 20 |
| PZG636 | | | | | | | | | 20 |
| PZG637 | C | | | | | | | | 25 |
| PZG638 | | | | | | | | | 30 |
| PZG639 | | | | | | L | | | 20 |
| PZG640 | | | | | | | | | 30 |
| PZG641 | C | | | | | | | | 15 |
| PZG642 | | | | T | | | | | 30 |
| PZG643 | | | | | | | | | 30 |
| PZG644 | H | | | | C | | W | | 30 |
| dCK1 | Y | D | E | I | Y | F | K | T | 80 |
| PZG645 | | | | | | | | | 30 |
| PZG646 | | | G | | | | | | 30 |
| PZG647 | | | | | | | | | 30 |
| PZG648 | | | | | | | | | 20 |
| PZG649 | | G | | | | | | | 30 |
| | * | | | | | * | | | |

### Example 8

### Site-Directed Mutagenesis to dCK1

Site directed mutagenesis deals with specific changes in the DNA sequence. Consequently, it is possible to alter the DNA sequence such that the coding sequence becomes changed. The result is a protein with amino acid changes. Mutations are generally targeted to the catalytic site of the protein with the purpose of increasing (or decreasing) the catalytic activity. Individual mutations made in the protein sequence and thereby in the structure can have vast consequences on the enzymatic activity. Site-directed mutagenesis is based on the extension of mismatched oligonucleotides, which incorporate point mutations into a new strand of DNA. The mutation introduced is amplified by PCR and the product can be cloned into a vector, followed by transformation and expression of the mutated gene.

Site-directed Mutagenesis of chicken dCK1. The mutant strand synthesis reaction was performed as recommended in the manual from Stratagene. A PCR reaction mixture (12.5 μl in total) was prepared on ice containing the following:

1.25 μl 10× reaction buffer from Stratagene

1 μl ds 10× diluted DNA template Chicken dCK (2-5ng)

0.1325 μl Primer 1 (forward mutation primer)

0.3125 μl Primer 2 (reverse mutation primer)

0.25 μl dNTP mix (Stratagene)

0.75 μl Quick solution

8.625 μl H₂O

and 0.25 μl 2.5 U/μl Pfu Turbo DNA polymerase (Stratagene) were mixed well with the reaction mixture.

A PCR program with the cycle parameters listed below was used:

Heated the reaction for 1 minute at 95° C. followed by 18 cycles of 50 seconds at 95° C.,

50 seconds at 60° C.

6 minutes at 68° C. (1 minute/kilobase of plasmid length)

final elongation at 68° C. for 7 minutes.

After the mutagenic PCR reaction, 2 μl were separated from the reaction mixture. The remaining sample reaction mixture was carefully mixed with 0.25 μl Dpnl (Stratagene), briefly centrifuged for 1 minute at 13000 rpm and incubated for at least 1 h at 37° C., as recommended in the instruction manual. Dpnl only cuts only methylated parental DNA template. 0.25 μl Dpnl digested and undigested (control) mix were transformed by electroporation into Top 10 cells. Upcoming colonies were grown on LB medium plus 100 μg/μl ampicillin overnight and containing Dpnl digested plamids with the mutated dCK1 gene in it, were inoculated in 10 ml LB+100 μg/μl amp overnight. The overnight culture was purified for plasmid. 10 μl from the purified plasmid were sent for sequencing to verify the plasmid borne genotype including the desired mutations.

The primers used for site directed mutagensis were:

P319 (Chicken dCK1 s-d.m. forward- primer: A94V,
R98M)

| | |
|---|---|
| 5′ CACCTTCCAGATGTACGTGTGCCTCAGCAT | (SEQ ID NO. 23) |
| GATTCGGGCTCAGCTC 3′ | |

P339 (Chicken dCK1 s-d.m. reverse- primer: A94V,
R98M)

| | |
|---|---|
| 5′ GAGCTGAGCCCGAATCATGCTGAGGCACAC | (SEQ ID NO. 24) |
| GTACATCTGGAAGGTG 3′ | |

P340 (Chicken dCK1 s-d.m. forward- primer: D127A)

| | |
|---|---|
| 5′ CTTTGAGCGATCTGTCTATAGTGCCAGATA | (SEQ ID NO. 25) |
| TATCTTTGCAGC 3′ | |

P341 (Chicken dCK1 s-d.m. reverse- primer: D127A)

| | |
|---|---|
| 5′ GCTGCAAAGATATATCTGGCACTATAGACA | (SEQ ID NO. 26) |
| GATCGCTCAAAG 3′ | |

P379 (Chicken dCK1 s-d.m. forward- primer: F90Y)

| | |
|---|---|
| 5′ GGTGGTCTTTCACCTACCAGATGTACGCGT | (SEQ ID NO. 27) |
| GCC 3′ | |

P380 (Chicken dCK1 s-d.m. reverse- primer: F90Y)

| | |
|---|---|
| 5′ GGCACGCGTACATCTG GTAGGTGA AA | (SEQ ID NO. 28) |
| GACCACC 3′ | |

Sabini et al. (2003) [Sabini, E., Ort, S., Monnerjahn, C., Konrad, M., and Lavie, A.: Structure of human dCK suggests strategies to improve anticancer and antiviral therapy. NatStruct.Biol. 10:513-519, 2003.) followed a strategy of mutating key active site residues in human dCK similar to those found in Dm-dNK. The result was a fourfold increase of efficiency toward gemcitabine and a fifty-fold increase of efficiency toward deoxycytidine. Due to the high homology between chicken (GgdCK1) and human dCK peptide sequence, it was of utmost interest to introduce the same mutations into the chicken dCK by site-directed mutagenesis as it was performed in human dCK. Introducing the determined mutations into the wild type sequence of chicken dCK to obtain the same triple mutant might lead to similar enhancement of activity as it was shown in human dCK.

The nucleotide positions, which were changed, to obtain the triple mutant in human and chicken dCK1 and the amino acid shift at the given position for both organisms are shown in Table 7. The difference of the positions, which were mutated to obtain the triple mutant can be explained due to a 6-amino acid insert (SCPSFS) behind position P7 in human dCK, which is lacking in the chicken peptide sequence.

TABLE 7
Overview over mutations introduced by site-directed mutagenesis.
Amino acid and nucleotide positions are shown, which are targeted
to obtain the “triple mutant” in human and chicken dCK1.

| | alaninie | arginine | asparagine |
|---|---|---|---|
| Targeted amino acid | (A) | (R) | (D) |
| Nucleotide sequence for aa in wt dCK1 | gcg | agg | gac |
| aa mutations in human dCK | A100V | R104M | D133A |
| aa mutations in chicken dCK | A94V | R98M | D127A |
| Mutated position in nucleotide sequence | 280 | 293 | 380 |
| Mutated nucleotide sequence | gtg | atg | gcc |
| in triple mutant | | | |

Mutations shown in table 7 were introduced into chicken wild type dCK1 by performing single site-directed mutagenesis twice. One pair of mutagenic primers (P340 and P341) introduced the mutation D127A and on top of this mutation, a second random mutagenesis reaction was performed afterwards with oligonucleotides P319 and P339, which introduced both mutations A94V and R98M at once. The individual mutants obtained by site directed mutagenesis were tested separately for their lethal dose value to investigate their effect on the activity of the dCK1 protein (Table 8).

The lethal dose values of the triple mutants from human and chicken were both found at around 25 nM (Table 8). The triple mutant from chicken showed a significant improvement of dCK1 activity as well, ca. 3-4-fold increase compared to wild type. The two mutants PZG689 and PZG691 did not show any improvement in activity, only when their mutations are combined. Comparing the improvement in dCK activity from wild type to triple mutants among human and chicken, one could find a slightly better rate of improvements in chicken, based on analysis of the lethal dose values.

TABLE 8
Screening results (LD₁₀₀) of chicken dCK1 mutants
obtained by site directed mutagenesis compared to
human dCK wild type and human dCK triple mutant.

| PZG | | | LD₁₀₀ |
|---|---|---|---|
| number | Origin and description | Mutation shift(s) | (nM) |
| 303 | human dCK wild type | none | 50-60 |
| 449 | human dCK triple mutant | A100V, R104M, | 25-30 |
| | | D133A | |
| 469 | chicken dCK1 wild type | none | 80 |
| 689 | chicken dCK1 one mutation | D127A | ca. 80 |
| 691 | chicken dCK1 two mutations | A94V, R98M | ca. 80 |
| 692 | chicken dCK1 triple mutant | A94V, R98M, D127A | 20-25 |

### Example 9

### Activity Measurements of Selected dCK1 Mutants

Activity measurements. Enzyme assays were done using radioactive labelled substrates as described previously. The data obtained (Table 9) from the activity assays describe the phosphorylation activity of the individual mutants toward either the natural substrate cytosine or its artificial analogue gemcitabine, respectively. The activity of gemcitabine compared to cytosine was found to be several-fold higher in every sample. dCK activity values based on cytosine as substrate were decreasingly small compared to the activity values based on gemcitabine. This can be explained since the colonies are screened and selected for gemcitabine. Extremely high differences in the activation values of gemcitabine compared to cytosine were found in PZG635 mutA and in PZG636 mutB, containing 6 and 1 amino acid mutations respectively. Both values showed more than a 1200-fold increase in activation. The ratio gemcitabine/cytosine from human wild type dCK and from the double gene (PZG634) show comparatively similar results to wild type values of chicken dCK1 in the range of approximately 150 to 430-fold. Only the sample PZG689, containing the mutation D127A in chicken dCK1 shows a poor increasing value of about 3 comparing the activation of gemcitabine with the activation of cytosine. That might lead to the assumption, that introducing this mutation D127A diminishes the activation of gemcitabine enormously. Cytosine and gemcitabine are concurring substrates to be metabolized by dCK. That leads to the statement, that the higher the ratio gemcitabine activation/cytosine activation is, the more improved is the actual mutant in respect to the objective of optimizing dCK as a suicide gene.

TABLE 9
Activity values in units (nmol/mg/min) of deoxycytidine and gemcitabine
and their ratios, obtained from activity assays of crude extracts
based on colonies carrying the plasmids listed below. The values
for chicken wild type are presented in bold.

| | | cytosine | gemcitabine | gemcita- |
|---|---|---|---|---|
| PZG | | [nmol/ | [nmol/ | bine/ |
| number | Description | mg/min] | mg/min] | cytosine |
| PZG303 | Hs dCK wt | 0.04 | 19.2 | 432 |
| PZG449 | Hs dCK triple mutant | 0.12 | 3.7 | 31 |
| PZG469 | Gg dCK 1 wt | 0.09 | 20.5 | 235 |
| PZG470 | Gg dCK 2 wt | 0.07 | 15.6 | 226 |
| PZG634 | Gg dCK double gene | 0.14 | 22.7 | 158 |
| PZG635 | Gg dCK mutA(6) | 0.09 | 120.5 | 1283 |
| PZG636 | Gg dCK mutB(1) | 0.09 | 117.4 | 1374 |
| PZG689 | Gg dCK D127A | 0.15 | 0.3 | 2 |
| PZG691 | Gg dCK A94V + R98M | 0.15 | 4.9 | 33 |
| PZG692 | Gg dCK triple mutant | 0.26 | 9.4 | 36 |

Numerous modifications and variations of the present invention are possible in light of the above teachings and, therefore, within the scope of the appended claims, the invention may be practiced otherwise than as particularly described.

1. (canceled)

2. An isolated polynucleotide according to claim 1, selected from the group consisting of:
(a) a polynucleotide encoding a polypeptide having the amino acid sequence of SEQ ID No. 2,
(b) a polynucleotide having the nucleotide sequence of SEQ ID No 1,
(c) a polynucleotide encoding a deoxycytidine kinase (dCK) polypeptide, said dCK polypeptide having at least 90% sequence identity to SEQ ID No2,
(d) a polynucleotide encoding a dCK polypeptide, said polynucleotide having at least 90% sequence identity to the coding sequence of SEQ ID No 1,
(e) a polynucleotide capable of hybridising to a complement of SEQ ID No. 1, said polynucleotide encoding a dCK,
(f) the complement of a through e,
(g) a polynucleotide encoding a polypeptide having the amino acid sequence of SEQ ID No. 4,
(h) a polynucleotide having the nucleotide sequence of SEQ ID No. 3
(i) a polynucleotide encoding a dCK polypeptide, said dCK polypeptide having at least 90% sequence identity to SEQ ID No. 4.
(j) a polynucleotide encoding a dCK polypeptide, said polynucleotide having at least 90% sequence identity to SEQ ID No. 3
(k) a polynucleotide capable of hybridising to a complement of SEQ ID No. 3 said polynucleotide encoding a dCK, and
(l) the complement of g through k.

3-6. (canceled)

7. The polynucleotide of claim 2, wherein the encoded polypeptide has at least 95% sequence identity to SEQ ID No 2.

8. The polynucleotide of claim 2, wherein the polynucleotide has at least 95% sequence identity to the coding sequence of SEQ ID No 1.

9. The polynucleotide of claim 2, wherein the polynucleotide encoding a dCK is capable of hybridising to a complement of SEQ ID No. 1 under conditions of at least medium stringency.

10. The polynucleotide of claim 2, wherein the encoded polypeptide has at least 95% sequence identity to SEQ ID No 4.

11. The polynucleotide of claim 2, wherein the polynucleotide has at least 95% sequence identity to the coding sequence of SEQ ID No 3.

12. The polynucleotide of claim 2, wherein the polynucleotide encoding a dCK is capable of hybridising to a complement of SEQ ID No. 3 under conditions of at least medium stringency.

13. The polynucleotide according to claim 2, wherein the encoded dCK comprises at least one mutation relative to the wildtype sequence at one or more of the positions in SEQ ID No. 2 or the corresponding position in SEQ ID No. 4: 4, 8, 11, 12, 49, 50, 54, 59, 60, 68, 71, 73, 74, 79, 82, 90, 92, 94, 98, 99, 103, 112, 115, 127, 139, 147, 156, 158, 177, 183, 184, 189, 190, 194, 204, 219, 239, and 247.

14. The polynucleotide according to claim 13, wherein the encoded dCK comprises one or more of the following mutations (positions corresponding to SEQ ID No. 2): P4L, E8G, E11G, G12D, A49V, R50G, V54A, E59G, E60G, S68P, S71I, G73R, N74S, M79T, K82E, K82R, F90Y, M92V, A94V, R98M, 199V, L103P, E112G, N115S, D127A, D139G, T147S, M156T, K158E, E177K, I183T, Y184C, Y184H, D189G, E190G, I194T, Y204C, F219C, F219L, K239W, and T247I.

15. The polynucleotide according to claim 14, wherein the encoded dCK comprises mutation(s) selected from the following group of mutations (SEQ ID No. 2 numbering):
F90Y;
E11G/K82E/199V/L103P;
E11G/G12D/A49V/N115S/F219C/T2471;
N74S;
E59G/M79T/Y184C;
E60G/G73R/K82R;
E11G/S71I/M92V/F219L;
P4L/E11G/T147S;
E11G/M79T/D139G/Y184C;
E8G/I194T;
S68P/K158E;
M156T/Y184H/Y204C/K239W;
E112G/I183T;
E11G/E190G;
V54A/E177K;
E11G/R50G;
P4L/D189G;
D127A;
A94V/R98M; and
A94V/R98M/D 127A.

16. The polynucleotide according to claim 14, wherein the encoded dCK comprises mutation(s) selected from the following group of mutations (SEQ ID No. 2 numbering): E11G/G12D/A49V/N115S/F219C/T247I and N74S.

17. The polynucleotide of claim 2, wherein the encoded dCK when compared to human Herpes simplex virus 1 (HSV-TK1) in a eukaryotic cell decreases at least four fold the LD₁₀₀ of at least one nucleoside analogue.

18. The polynucleotide of claim 17, wherein the LD₁₀₀ is decreased at least 10 fold.

19. A vector comprising the nucleic acid of claim 2.

20. The vector of claim 19, being an expression vector.

21. The expression vector of claim 20, being a viral vector, such as a Herpes simplex viral vector, an adenoviral vector (in particular an oncolytic adenovirus), an adeno-associated viral vector, a lentiviral vector, a retroviral vector.

22. An isolated host cell genetically engineered with the vector of claim 19.

23. The isolated host cell of claim 22, being a prokaryotic cell.

24. The isolated host cell of claim 22, which is a eukaryotic cell.

25. The host cell of claim 24, being selected from the group consisting of human stem cells, and human precursor cells.

26. A packaging cell line capable of producing an infective virion comprising the virus vector of claim 21.

27. A process for producing a dCK polypeptide comprising culturing a host cell of claim 22 in vitro and recovering the expressed dCK from the culture.

28. (canceled)

29. An isolated deoxycytidine kinase polypeptide comprising a polypeptide selected from the group consisting of:
(a) a polypeptide having the amino acid sequence of SEQ ID No 2,
(b) a dCK polypeptide having at least 90% sequence identity to SEQ ID No2,
(c) a polypeptide having the amino acid sequence of SEQ ID No. 4, and
(d) a dCK polypeptide having at least 90% sequence identity to SEQ ID No. 4.

30. (canceled)

31. The polypeptide of claim 29, wherein the polypeptide has at least 95% sequence identity to SEQ ID No 2.

32. The polypeptide of claim 29, wherein the polypeptide has at least 95% sequence identity to SEQ ID No 4.

33. The isolated deoxycytidine kinase polypeptide of claim 29, comprising at least one mutation relative to the wildtype sequence at one or more of the positions in SEQ ID No. 2 or the corresponding position in SEQ ID No. 4: 4, 8, 11, 12, 49, 50, 54, 59, 60, 68, 71, 73, 74, 79, 82, 90, 92, 94, 98, 99, 103, 112, 115, 127, 139, 147, 156, 158, 177, 183, 184, 189, 190, 194, 204, 219, 239, and 247.

34. The isolated deoxycytidine kinase polypeptide of claim 29, comprising one or more of the following mutations (positions corresponding to SEQ ID No. 2): P4L, E8G, E11G, G12D, A49V, R50G, V54A, E59G, E60G, S68P, S71I, G73R, N74S, M79T, K82E, K82R, F9OY, M92V, A94V, R98M, I99V, L103P, E112G, N115S, D127A, D139G, T147S, M156T, K158E, E177K, I183T, Y184C, Y184H, D189G, E190G, I194T, Y204C, F219C, F219L, K239W, and T247I.

35. The isolated deoxycytidine kinase polypeptide of claim 34, comprising mutation(s) selected from the following group of mutations (SEQ ID No. 2 numbering):
F90Y;
E11G/K82E/199V/L103P;
E11G/G12D/A49V/N115S/F219C/T247I;
N74S;
E59G/M79T/Y184C;
E60G/G73R/K82R;
E11G/S71I/M92V/F219L;
P4L/E11G/T147S;
E11G/M79T/D139G/Y184C;
E8G/I194T;
S68P/K158E;
M156T/Y184H/Y204C/K239W;
E112G/I183T;
E11G/E190G;
V54A/E177K;
E11G/R50G;
P4L/D189G;
D127A;
A94V/R98M; and
A94V/R98M/D 127A.

36. The isolated deoxycytidine kinase polypeptide of claim 34, comprising mutation(s) selected from the following group of mutations (SEQ ID No. 2 numbering): E11G/G12D/A49V/N115S/F219C/T247I and N74S.

37. The isolated deoxycytidine kinase polypeptide of claim 29, which dCK when compared to human Herpes simplex virus 1 (HSV-TK1) in a eukaryotic cell decreases at least four fold the LD₁₀₀ of at least one nucleoside analogue.

38. The isolated dCK of claim 37, wherein the LD₁₀₀ is decreased at least 10 fold.

39. A pharmaceutical composition comprising the polypeptide of claim 29 and a pharmaceutically acceptable carrier or diluent.

40. A pharmaceutical composition comprising the expression vector of claim 20 and a pharmaceutically acceptable carrier or diluent.

41. A pharmaceutical composition comprising the host cell of claim 22 and optionally a pharmaceutically acceptable carrier or diluent.

42. A pharmaceutical composition comprising the packaging cell line of claim 26, and optionally a pharmaceutically acceptable carrier or diluent.

43-48. (canceled)

49. Pharmaceutical articles comprising a source of a Gallus gallus derived dCK or a functional analog thereof and a nucleoside analogue for the simultaneous, separate or successive administration in treatment of a pathogenic agent.

50. Articles according to claim 49, wherein the pathogenic agent is a tumour cell.

51. Articles according to claim 49, wherein the pathogenic agent is a virus, a bacterium or a parasite.

52. Articles according to claim 49, wherein the nucleoside analogue is a cytidine analogue.

53. Articles according to claim 49, wherein the nucleoside analogue is gemcitabine.

54. Articles according to claim 49, wherein the nucleoside analogue is Ara-G.

55. Articles according to claim 49, wherein the nucleoside analogue is selected from the group consisting of D4T, ddC, AZT, ACV, 3TC, ddA, fludarabine, Cladribine, araC, gemcitabine, Clofarabine, Nelarabine (araG) and Ribarivin.

56. Articles according to claim 49, wherein the source of dCK comprises the polypeptide of claim 29.

57. Articles according to claim 49, wherein the source of dCK comprises the expression vector of claim 20.

58. Articles according to claim 49, wherein the source of dCK comprises the host cell of claim 22.

59. Articles according to claim 49, wherein the source of dCK comprises the packaging cell line of claim 26.

60. A method of sensitising a cell to a nucleoside analogue prodrug, which method comprises the steps of:
(i) transfecting or transducing said cell with a polynucleotide sequence of claim 2 encoding a deoxycytidine kinase enzyme capable of promoting the conversion of said prodrug into a cytotoxic drug, or with an expression vector comprising said polynucleotide sequence; and
(ii) delivering said nucleoside analogue prodrug to said cell;
wherein said cell is more sensitive to said cytotoxic drug than to said nucleoside analogue prodrug.

61. The method of claim 60, wherein the nucleoside analogue is selected from the group consisting of D4T, ddC, AZT, ACV, 3TC, ddA, fludarabine, Cladribine, araC, gemcitabine, Clofarabine, Nelarabine (araG) and Ribarivin.

62. The method of claim 60, wherein said nucleoside analogue prodrug is a cytidine analogue.

63. The method of claim 60, wherein said nucleoside analogue prodrug is gemcitabine.

64. The method of claim 60, wherein said nucleoside analogue prodrug is Ara-G.

65. A method of inhibiting a pathogenic agent in a warm-blooded animal, which method comprises administering to said animal a polynucleotide of claim 2 or expression vector of any claim 20.

66. The method of claim 65, wherein said polynucleotide or said expression vector is administered in vivo.

67. The method of claim 65, wherein said pathogenic agent is a virus, a bacterium, or a parasite.

68. The method of claim 65, wherein said pathogenic agent is a tumour cell.

69. The method of claim 65, wherein said pathogenic agent is an autoreactive immune cell.

70. The method of claim 65, further comprising the step of administering a nucleoside analogue to said warm-blooded animal.

71. The method of claim 70, wherein the nucleoside analogue is selected from the group consisting of D4T, ddC, AZT, ACV, 3TC, ddA, fludarabine, Cladribine, araC, gemcitabine, Clofarabine, Nelarabine (araG) and Ribarivin.

72. The method of claim 70, wherein said nucleoside analogue is a cytidine analogue.

73. The method of claim 72, wherein said nucleoside analogue is gemcitabine.

74. The method of claim 72, wherein said nucleoside analogue is Ara-G.

75. An antibody against the polypeptide of claim 29.

76. A method of phosphorylating a nucleoside or nucleoside analogue comprising the steps of.
(i) subjecting the nucleoside or nucleoside analogue to the action of the Gallus gallus dCK of claim 29, and
(j) recovering the phorphorylated nucleoside or nucleoside analogue.

77. The method of claim 76, wherein the nucleoside or nucleoside analogue is a cytidine or a cytidine analogue.

78. A method for the treatment of a patient having need of dCK comprising: administering to the patient a therapeutically effective amount of the polypeptide of claim 29, wherein the polypeptide is administered by providing to the DNA encoding said polypeptide and expressing said polypeptide in vivo.

79. A process for identifying compounds effective as antagonists to Gallus gallus dCKs comprising combining the polypeptide of claim 29, a compound to be screened and a reaction mixture containing deoxyribonucleosides; and determining the ability of the compound to inhibit the phosphorylation of the deoxyribonucleosides.

80. A method of non-invasive nuclear imaging of transgene expression of a chicken deoxycytidine kinase enzyme of the invention in a cell or subject, which method comprises the steps of
(i) transfecting or transducing said cell or subject with a polynucleotide sequence encoding the deoxycytidine kinase enzyme of claim 29, which enzyme promotes the conversion of a substrate into a substrate-monophosphate;
(ii) delivering said substrate to said cell or subject; and
(iii) non-invasively monitoring the change to said prodrug in said cell or subject.

81. The method of claim 80, wherein the monitoring carried out in step (iii) is performed Single Photon Emission Computed Tomography (SPECT), by Positron Emission Tomography (PET), by Magnetic Resonance Spectroscopy (MRS), by Magnetic Resonance Imaging (MRI), or by Computed Axial X-ray Tomography (CAT), or a combination thereof.

82. The method of either of claims 80 to 81, wherein the substrate is a labelled nucleoside analogue.

83. An isolated eukaryotic deoxyadenosine kinase (dAK) enzyme (EC 2.7.1.76).

84. The dAK enzyme of claim 83, derived from a vertebrate.

85. The dAK enzyme of claim 83, derived from an avian species.

86. The dAK enzyme of claim 83, derived from a fish.

87. The dAK enzyme of claim 83, derived from a reptile.

88. The dAK enzyme of claim 83, derived from an amphibian species.

89. The dAK enzyme of claim 83, selected from the group consisting of:
a. a dAK enzyme having the amino acid sequence of SEQ ID No. 4,
b. a dAK enzyme having an amino acid sequence that is at least 90% sequence identity to SEQ ID No. 4,
c. a dAK enzyme encoded by a polynucleotide having at least 90% sequence identity to SEQ ID No. 3, and
d. a dAK enzyme encoded by a polynucleotide capable of hybridising to a polynucleotide having the complement of SEQ ID No. 3.

90. The dAK enzyme of claim 83, wherein the dAK has an amino acid sequence that has at least 95% sequence identity to SEQ ID No 4.

91. The dAK enzyme of claim 83, wherein the dAK has an amino acid sequence that has at least 95% sequence identity to SEQ ID No 34.

92. The dAK enzyme of claim 83, wherein the dAK has an amino acid sequence that has at least 90% sequence identity to SEQ ID No 35.

93. The dAK enzyme of claim 83, the amino acid sequence of which in a multisequence alignment using Clustal W 1.81 forms a phylogenetic sub-group together with Chicken dAK (SEQ ID No. 4) and distinct from GgdCK1 (SEQ ID No. 2), human dCK (SEQ ID No. 31), rat dCK (SEQ ID No. 33), and mouse dCK (SEQ ID No 32.).

94. A polynucleotide encoding a dAK enzyme of claim 83.

95. A vector comprising the polynucleotide sequence of claim 94.

96. A host cell transfected or transduced with the vector of claim 95.

97. A pharmaceutical composition comprising the dAK enzyme of claim 83, the polynucleotide of claim 94, the vector of claim 95, or the host cell of claim 96, and a pharmaceutically acceptable excipient, diluent or carrier.

98. The pharmaceutical composition of claim 97, further comprising at least one nucleoside analogue, preferably wherein said nucleoside analogue is a adenosine analogue, more preferably ara-G, fludarabine or cladribine for the successive, simultaneous or separate administration.

99-100. (canceled)

101. A method of sensitising a cell to a nucleoside analogue prodrug, which method comprises the steps of:
(i) transfecting or transducing said cell with a polynucleotide sequence of claim 94 encoding a deoxycytidine kinase enzyme capable of promoting the conversion of said prodrug into a cytotoxic drug, or with an expression vector comprising said polynucleotide sequence; and
(ii) delivering said nucleoside analogue prodrug to said cell;
wherein said cell is more sensitive to said cytotoxic drug than to said nucleoside analogue prodrug.
